(19) United States
(12) Patent Application Publication
(10) Pub. No.: US 2019/0381137 A1
(43) Pub. Date: Dec. 19, 2019

(54) BOLAAMPHIPHILIC COMPOUNDS, COMPOSITIONS AND USES THEREOF

(71) Applicant: Lauren Sciences LLC, New York, NY (US)

(72) Inventors: Charles LINDER, Rehovot (IL); Eliahu HELDMAN, Rehovot (IL); Sarina GRINBERG, Meitar (IL)

(21) Appl. No.: 16/454,537

(22) Filed: Jun. 27, 2019

Related U.S. Application Data
(63) Continuation of application No. 15/655,338, filed on Jul. 20, 2017, now abandoned.
Application No. 15/655,338 is a division of application No. 14/638,437, filed on Mar. 4, 2015, now abandoned.
Application No. 14/638,437 is a continuation of application No. PCT/US2013/057955, filed on Sep. 4, 2013.
(60) Provisional application No. 61/696,790, filed on Sep. 4, 2012.
(60) Provisional application No. 62/065,160, filed on Oct. 17, 2014.

(51) Int. Cl. A61K 38/18; A61K 47/64; A61K 49/00; A61K 31/713

(52) U.S. Cl. CPC A61K 38/185; A61K 31/713; A61K 49/0082; A61K 47/6455

(57) ABSTRACT

Bolaamphiphilic compounds are provided according to formula I:

HG²-L¹-HG¹ I

where HG¹, HG² and L¹ are as defined herein. Provided bolaamphilphilic compounds and the pharmaceutical compositions thereof are useful for delivering siRNA into animal or human cell.

### CROSS REFERENCE TO RELATED APPLICATIONS

This application is a continuation of U.S. application Ser. No. 15/655,338 filed Jul. 20, 2017, which is a divisional of U.S. application Ser. No. 14/638,437 filed Mar. 4, 2015, now abandoned, which is a continuation of International Application No. PCT/US2013/057955, filed Sep. 4, 2013, which claims priority to U.S. Application No. 61/696,790, filed Sep. 4, 2012, the contents of each of which are incorporated by reference herein. U.S. application Ser. No. 14/638,437, filed Mar. 4, 2015, further claims the benefit of U.S. Application No. 62/065,160 filed Oct. 17, 2014, the contents of which are also incorporated by reference herein.

### FIELD

Provided herein are bolaamphiphilic compounds, complexes thereof with specific small interfering RNAs (siRNAs), and pharmaceutical compositions thereof. Also provided are methods of delivering siRNAs into the human and animal cell using the compounds, complexes and pharmaceutical compositions provided herein. Also provided are methods of delivering siRNAs into the human and animal organs, such as the brain, using the compounds, complexes and pharmaceutical compositions provided herein.

### BACKGROUND

In the past decade, efforts to develop RNA-based therapeutic technologies have significantly intensified¹⁻⁵. Triggering RNA interference (RNAi), in particular, has become one of the most widely used techniques for biomedical applications¹⁻¹¹. RNAi employs a mechanism of post-transcriptional sequence specific gene silencing by processing double-stranded RNAs into small-interfering RNAs (siRNAs) used as part of the RNA-induced silencing complex (RISC) to selectively cleave target mRNA¹². After the discovery that synthetic siRNAs can be exogenously introduced into cells to activate RNAi^{13, 14}, this approach has become a powerful method for selective suppression of specific genes of interest in different species, showing potential for use in cancer therapeutics^{3, 5, 6}. However, the biomedical utility of the synthetic siRNAs is limited by several RNA structure-related factors such as the negative charge (uptake by cells that also have negatively charged surface) and instability in the blood circulation (non-modified siRNAs have a very short half-life in blood stream, mostly because of degradation by nucleases)⁴. These impediments can be overcome by using polymeric or lipid-based carriers to shield the negative charge and provide protection against nuclease activity¹⁵⁻¹⁷.

Complexation of the anionic carboxyfluorescein (CF) with single headed amphiphiles of opposite charge in cationic vesicles, formed by mixing single-tailed cationic and anionic surfactants has been reported (Danoff et al. 2007).

Furthermore, WO 02/055011 and WO 03/047499, both of the same applicant, disclose amphiphilic derivatives composed of at least one fatty acid chain derived from natural vegetable oils such as vernonia oil, lesquerella oil and castor oil, in which functional groups such as epoxy, hydroxy and double bonds were modified into polar and ionic headgroups.

Additionally, WO 10/128504 discloses a series of amphiphiles and bolamphiphiles (amphiphiles with two head groups) useful for targeted drug delivery of insulin, insulin analogs, TNF, GDNF, DNA, RNA (including siRNA), enkephalin class of analgesics, and others.

These synthetic bolaamphiphiles (bolas) have recently been shown to form nanovesicles that interact with and encapsulate a variety of small and large molecules including peptides^{18, 19} proteins²⁰ and plasmid DNAs^{19, 21} delivering them across biological membranes²². These bolaamphiphiles are a unique class of compounds that have two hydrophilic headgroups placed at each ends of a hydrophobic domain. Bolaamphiphiles can form vesicles that consist of monolayer membrane that surrounds an aqueous core. Vesicles made from natural bolaamphiphiles, such as those extracted from archaebacteria (archaesomes), are very stable and, therefore, might be employed for targeted drug delivery. However, bolaamphiphiles from archaebacteria are heterogeneous and cannot be easily extracted or chemically synthesized. Furthermore, bolas have a hydrophobic alkyl chain connected to positively charged head groups, that can potentially interact with negatively charged nucleic acids and promote their delivery into cells. However, the nature of these interactions as well as the possibility to use bolas for optimized delivery of therapeutic siRNAs remains a challenge.

Thus, there remains a need to make new specific bolaamphiphiles which can be useful for optimized delivery of siRNAs into cells and have desired therapeutic utility. The compounds, compositions, and methods described herein are directed toward this end.

### SUMMARY OF THE INVENTION

In certain aspects, provided herein are pharmaceutical compositions comprising of complexes between bolaamphiphiles and pharmacologically or biologically active compounds.

In certain aspects, the bolaamphiphile vesicle complexes comprise one or more bolaamphiphilic compounds and the biologically active compound is siRNA.

In certain aspects, the bolaamphiphile vesicle complexes comprise one or more bolaamphiphilic compounds and the biologically active compound is a siRNA that is a mixture of two or more siRNA, wherein at least one siRNA is directed to a first target, and at least one siRNA is directed to a second target.

In further aspects, provided herein are novel siRNA and bolamphiphilic vesicle complex comprising siRNA and one or more bolaamphiphilic compounds.

In further aspects, provided herein are novel formulations of siRNA with bolaamphiphilic compounds or with bolaamhphilic vesicles.

In another aspect, provided here are methods of delivering siRNA into animal or human cells.

In an additional aspect, this present disclosure is directed to delivery of siRNA-bolaamphiphile vesicle complexes or siRNA-bolaamphiphilic vesicle complexes into animals or human wherein the bolaamphiphile vesicle complex comprises one or more bolaamphiphilic compounds and siRNA.

In another aspect, provided herein are methods of delivering siRNA into animal or human cell comprising the step of administering to the animal or human a pharmaceutical composition comprising of a bolaamphiphile vesicle complex; and wherein the bolaamphiphile vesicle complex comprises one or more bolaamphiphilic compounds and siRNA. In one embodiment, the cell is brain cell, liver cell, gall bladder, or a lung cell. In other embodiments, the cells are are cells of a lymph node, a CD4+ lymphocyte, or a cell of the mononuclear phagocyte system, including, without limitation, a monocyte, macrophage, a resident brain microglial cell and a dendritic cell. In a still further emobidment, the cell is a cancer cell.

In another aspect, provided here are methods of delivering siRNA into animal or human organs comprising the step of administering to the animal or human a pharmaceutical composition comprising of a bolaamphiphile vesicle complex; and wherein the bolaamphiphile vesicle complex comprises one or more bolaamphiphilic compounds and siRNA. In one embodiment, the organ is brain, liver, gall bladder, a lymph node or a lung. In certain aspects of this emobidment, the siRNA is delivered to a tumor.

In a further embodiment the active agent is an RNA-DNA heteroduplex with properties of siRNA molecules. In certain aspects of this embodiment, the bolaamphiphile vesicle complexes comprise one or more bolaamphiphilic compounds and the biologically active compound is a siRNA that is a mixture of two or more siRNA or a mixture comprising at least one siRNA and one RNA-DNA duplex, wherein at least one siRNA or RNA-DNA duplex is directed to a first target, and at least one siRNA or RNA-DNA duplex is directed to a second target.

In certain embodiments, the target is a promoter. In other emodiments, the first and second targets are sequences of separate and distinct genes.

In other embodments, the bolaamphiphile vesicle complexes disclosed comprise one or more bolaamphiphilic compounds and one or more biologically active compounds selected from among basic amino acids (e.g., histidine), mRNA molecules, antisense oligonucleotides, and peptide targeting ligands.

The present disclosure is further directed to methods of delivering bolaamphiphile vesicle complexes disclosed comprise one or more bolaamphiphilic compounds and one or more biologically active compounds selected from among basic amino acids (e.g., histidine), mRNA molecules, antisense oligonucleotides, and peptide targeting ligands

In another aspect, provided herein are methods for delivering basic amino acids (e.g., histidine), mRNA molecules, antisense oligonucleotides, and peptide targeting ligands into animal or human organs comprising the step of administering to the animal or human a pharmaceutical composition comprising a bolaamphiphile vesicle complex; and wherein the bolaamphiphile vesicle complex comprises one or more bolaamphiphilic compounds and a biologically active compound selected from among basic amino acids (e.g., histidine), mRNA molecules, antisense oligonucleotides, peptide targeting ligands and combinations thereof. In one embodiment, the organ is brain, liver, gall bladder, a lymph node or a lung. In certain aspects of this emobidment, the biologically active compound, selected from among basic amino acids (e.g., histidine), mRNA molecules, antisense oligonucleotides, peptide targeting ligands and combinations thereof, is delivered to a tumor. In other aspects of this embodiment, the compositions are delivered to other organs, tissue, and cells as described hererin.

In one embodiment, the bolaamphiphilic compound consists of two hydrophilic headgroups linked through a long hydrophobic chain. In another embodiment, the hydrophilic headgroup is an amino containing group. In a specific embodiment, the hydrophilic headgroup is a tertiary or quaternary amino containing group.

In one particular embodiment, the bolaamphiphilic compound is a compound according to formula I:

HG²-L¹-HG¹ I

or a pharmaceutically acceptable salt, solvate, hydrate, prodrug, stereoisomer, tautomer, isotopic variant, or N-oxide thereof, or a combination thereof wherein:

each HG¹ and HG² is independently a hydrophilic head group; and

L¹ is alkylene, alkenyl, heteroalkylene, or heteroalkenyl linker; unsubstituted or substituted with C₁-C₂₀ alkyl, hydroxyl, or oxo.

In one embodiment, the pharmaceutically acceptable salt is a quaternary ammonium salt.

In one embodiment, with respect to the bolaamphiphilic compound of formula I, the bolaamphiphilic compound is a compound according to formula II, III, IV, V, or VI:

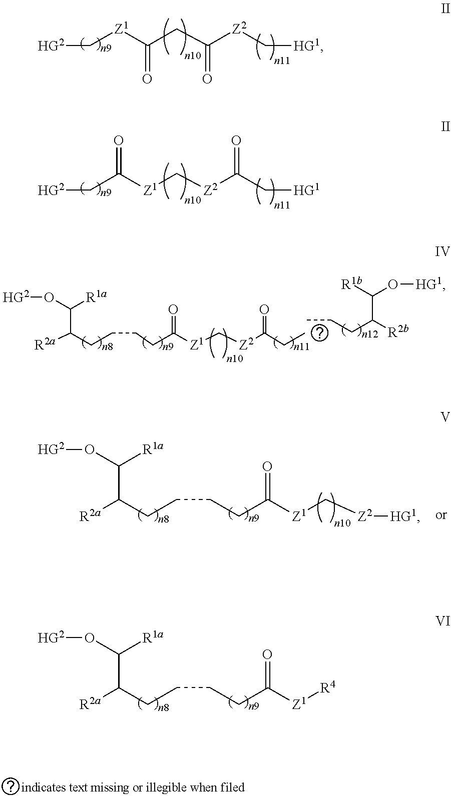

or a pharmaceutically acceptable salt, solvate, hydrate, prodrug, stereoisomer, tautomer, isotopic variant, or N-oxide thereof, or a combination thereof;
wherein:

each HG¹ and HG² is independently a hydrophilic head group;

each Z¹ and Z² is independently —C(R³)₂—, —N(R³)— or —O—;

each R^{1a}, R^{1b}, R³, and R⁴ is independently H or C₁-C₈ alkyl;

each R^{2a} and R^{2b} is independently H, C₁-C₈ alkyl, OH, alkoxy, or O-HG¹ or O-HG²;

each n8, n9, n11, and n12 is independently an integer from 1-20;

n10 is an integer from 2-20; and

each dotted bond is independently a single or a double bond.

In one embodiment, with respect to the bolaamphiphilic compound of formula I, II, III, IV, V, or VI, each HG¹ and HG² is independently selected from:

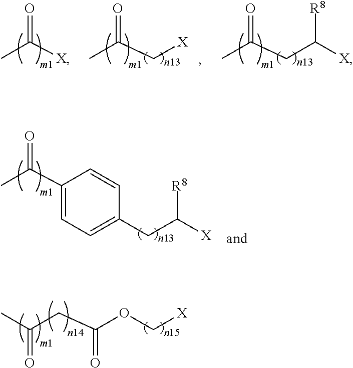

wherein:
X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocyclyl;
each R^{5c} is independently substituted or unsubstituted C₁-C₂₀ alkyl; each R⁸ is independently H, substituted or unsubstituted C₁-C₂₀ alkyl, alkoxy, or carboxy;
m1 is 0 or 1; and
each n13, n14, and n15 is independently an integer from 1-20.

In one embodiment, with respect to the bolaamphiphilic compound of formula I, the bolaamphiphilic compound is selected from the bolaambphilic compounds listed in Table 1, and wherein the compound ID is GLH-7, GLH-9, GLH-10, GLH-11, GLH-14, GLH-15, GLH-17, GLH-18, GLH-22, GLH-23, GLH-24, GLH-25, GLH-27, GLH-28, GLH-30 to GLH-48, GLH-55, GLH-56, or GLH-57.

Other objects and advantages will become apparent to those skilled in the art from a consideration of the ensuing detailed description.

### FIGURES

FIG. 1: Double stranded siRNA and fluorescently labeled siRNA used for the in vitro and the in vivo experiments

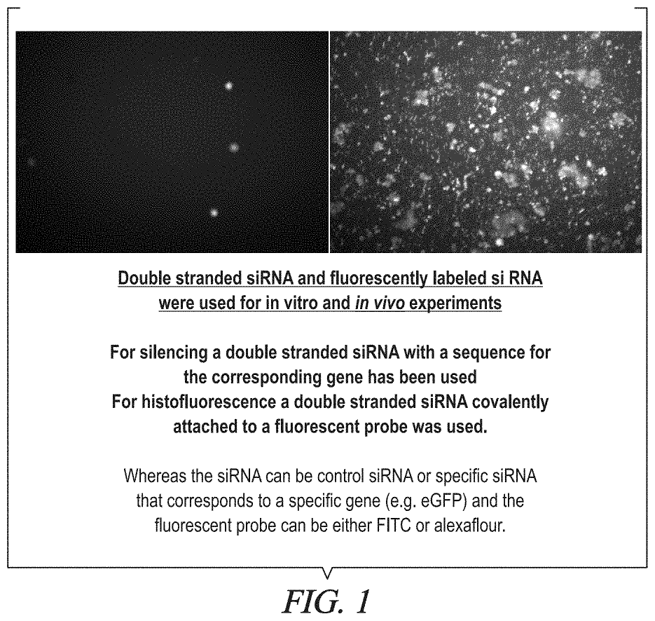

FIG. 2: Transfection of FITC-siRNA into dendritic cells and silencing of HSP60 gene in these cells by bolaamphiphilic vesicles with siRNA. Human primary dendritic cells (obtained by differentiating human monocytes by the cytokines IL-4 and GM-CSF) were exposed to GLH-19 vesicles with FITC-siRNA for 5 hours and cells were observed by a fluorescence microscope and examined by flow cytometry. The fluorescence micrographs show that all the cells became fluorescent after exposing them for 5 hours to bolaamphiphilic vesicles that contained FITC-siRNA whereas only few cells were fluorescent after transfecting them with the same concentration of FITC-siRNA by electroporation. These results were confirmed by flow cytometry studies shown in the lower part of the Figure. When the dendritic cells were exposed to GLH-19 vesicles with specific siRNA for HSP60, a very significant silencing of the gene was seen on a western blot compared to cells treated with empty vesicles (control cells). (V-smart vesicles−bolaamphiphilic vesicles).

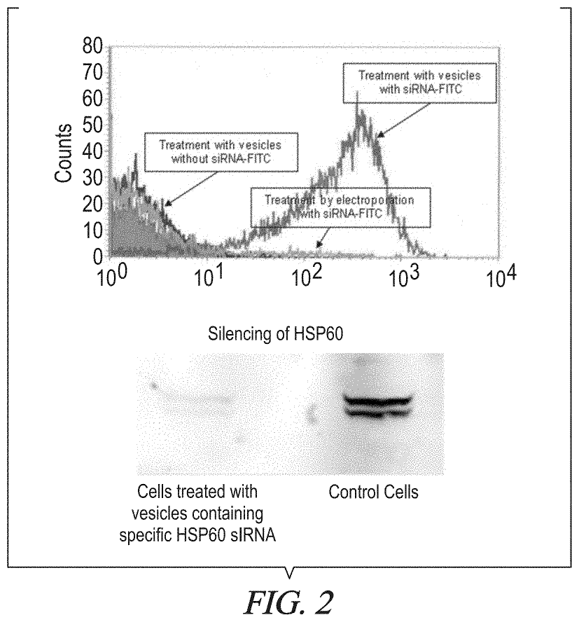

FIG. 3: Silencing of GFP gene in stably transfected macorophages by GFP siRNA. Macrophages cell line from a mouse that stably express eGFP were exposed to GLH-19 vesicles containg eGFP-siRNA. Not all the cells expressed the GFP as can be seen from comparison of the phase contrast micrograph to the fluorescence micrograph of untreated cells (lower right micrograph and upper right micrographs, respectively). Yet, the eGFP fluorescence in the cells that expressed the GFP gene disappeared almost completely when the cells were treated with GLH-19 vesicles containing eGFP-siRNA (lower left micrograph), whereas in cells that were treated with empty vesicles all the cells that expressed the eGFP remained fluorescent (upper left micrograph) (V-smart vesicles−bolaamphiphilic vesicles).

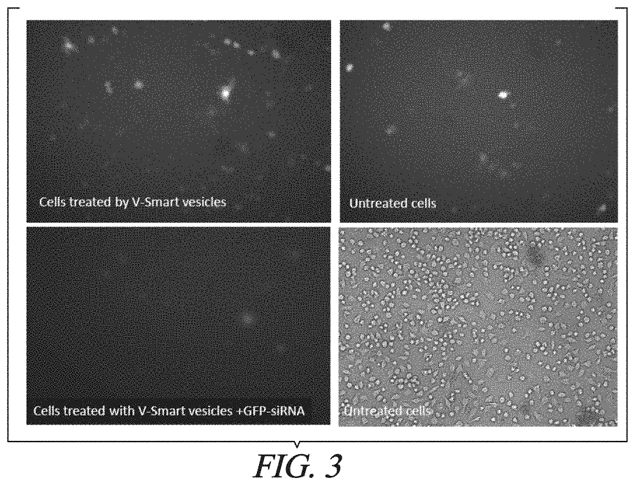

FIG. 4: Silencing of eGFP in MDA-MB-231/GFP cell line treated with GLH-19 vesicles containing eGFP-siRNA. The breast cancer cell line MDA-MB-231 that stably express eGFP were exposed for 5 hours to GLH-19 vesicles containing egFP-siRNA. 72 hours after the exposure a very significant silencing of the eGFP gene was observed. No silencing was seen in cells exposed to empty vesicles (not shown).

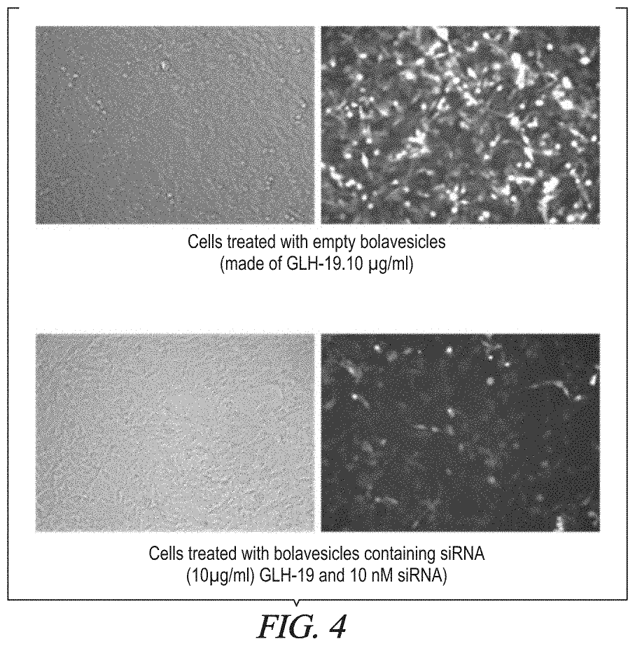

FIG. 5: Biodistribution of siRNA-AF555 in organs from mice after i.v. administration of bolavesicles containing siRNA-AF555. GLH-19 vesicles containing AF555-siRNA were injected via the tail vein into mice and 30 minutes later mice were acrificed, organs collected and imaging for AF-555 fluorescence was performed. Bakground fluorescence was adjusted to show brown color and AF-555 fluorescence was adjusted to give green color. AF-555 fluorescence is seen only in gall bladder (left image), blood vessels (around the lung, see middle image) and thoughout the brain (right image). No fluorescence is seen in the liver at the 30 min time point nor in the lung.

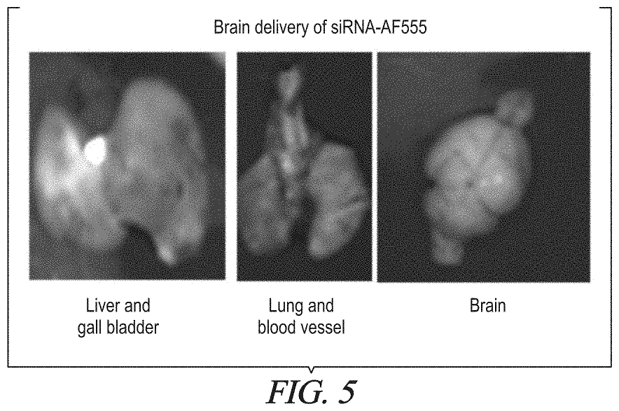

### DEFINITIONS

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The following terms are intended to have the meanings presented therewith below and are useful in understanding the description and intended scope of the present invention. When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e. at least one) of the grammatical objects of the article. By way of example “an analogue” means one analogue or more than one analogue.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl.

“Alkylene” refers to a substituted or unsubstituted alkyl group, as defined above, wherein two hydrogens are removed to provide a divalent radical. Exemplary divalent alkylene groups include, but are not limited to, methylene (—CH₂—), ethylene (—CH₂CH₂—), the propylene isomers (e.g., —CH₂CH₂CH₂— and —CH(CH₃)CH₂—) and the like.

“Alkenyl” refers to a radical of a straight-chain or branched hydrocarbon group having from 2 to 20 carbon atoms, one or more carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkenylene” refers a substituted or unsubstituted alkenyl group, as defined above, wherein two hydrogens are removed to provide a divalent radical. Exemplary divalent alkenylene groups include, but are not limited to, ethenylene (—CH═CH—), propenylenes (e.g., —CH═CHCH₂— and —C(CH₃)═CH— and —CH═C(CH₃)—) and the like.

“Alkynyl” refers to a radical of a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

“Alkynylene” refers a substituted or unsubstituted alkynyl group, as defined above, wherein two hydrogens are removed to provide a divalent radical. Exemplary divalent alkynylene groups include, but are not limited to, ethynylene, propynylene, and the like.

“Aryl” refers to a radical of a monocyclic or polycyclic (e.g., bicyclic or tricyclic) 4n+2 aromatic ring system (e.g., having 6, 10, or 14% electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

In certain embodiments, an aryl group substituted with one or more of groups selected from halo, C₁-C₈ alkyl, C₁-C₈ haloalkyl, cyano, hydroxy, C₁-C₈ alkoxy, and amino.

Examples of representative substituted aryls include the following

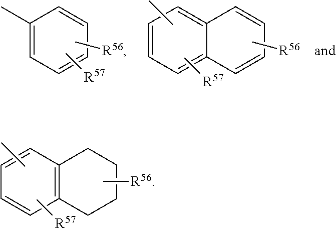

In these formulae one of R⁵⁶ and R⁵⁷ may be hydrogen and at least one of R⁵⁶ and R⁵⁷ is each independently selected from C₁-C₈ alkyl, C₁-C₈ haloalkyl, 4-10 membered heterocyclyl, alkanoyl, C₁-C₈ alkoxy, heteroaryloxy, alkylamino, arylamino, heteroarylamino, NR⁵⁸COR⁵⁹, NR⁵⁸SOR⁵⁹NR⁵⁸SO₂R⁵⁹, COOalkyl, COOaryl, CONR⁵⁸R⁵⁹, CONR⁵⁸OR⁵⁹, NR⁵⁸R⁵⁹, SO₂NR⁵⁸R⁵⁹, S-alkyl, SOalkyl, SO₂alkyl, Saryl, SOaryl, SO₂aryl; or R⁵⁶ and R⁵⁷ may be joined to form a cyclic ring (saturated or unsaturated) from 5 to 8 atoms, optionally containing one or more heteroatoms selected from the group N, O, or S. R⁶⁰ and R⁶¹ are independently hydrogen, C₁-C₈ alkyl, C₁-C₄ haloalkyl, C₃-C₁₀ cycloalkyl, 4-10 membered heterocyclyl, C₆-C₁₀ aryl, substituted C₆-C₁₀ aryl, 5-10 membered heteroaryl, or substituted 5-10 membered heteroaryl.

“Fused aryl” refers to an aryl having two of its ring carbon in common with a second aryl ring or with an aliphatic ring.

“Aralkyl” is a subset of alkyl and aryl, as defined herein, and refers to an optionally substituted alkyl group substituted by an optionally substituted aryl group.

“Heteroaryl” refers to a radical of a 5-10 membered monocyclic or bicyclic 4n+2 aromatic ring system (e.g., having 6 or 10% electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following:

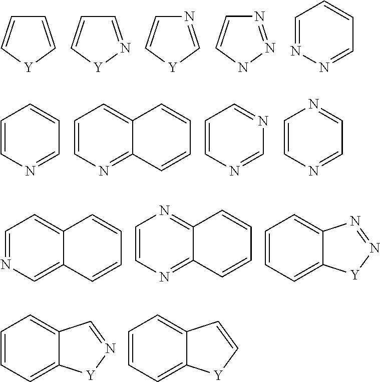

wherein each Y is selected from carbonyl, N, NR⁶⁵, O, and S; and R⁶⁵ is independently hydrogen, C₁-C₈ alkyl, C₃-C₁₀ cycloalkyl, 4-10 membered heterocyclyl, C₆-C₁₀ aryl, and 5-10 membered heteroaryl.

Examples of representative aryl having hetero atoms containing substitution include the following:

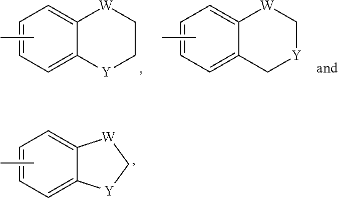

wherein each W is selected from C(R⁶⁶)₂, NR⁶⁶, O, and S; and each Y is selected from carbonyl, NR⁶⁶, O and S; and R⁶⁶ is independently hydrogen, C₁-C₈ alkyl, C₃-C₁₀ cycloalkyl, 4-10 membered heterocyclyl, C₆-C₁₀ aryl, and 5-10 membered heteroaryl.

“Heteroaralkyl” is a subset of alkyl and heteroaryl, as defined herein, and refers to an optionally substituted alkyl group substituted by an optionally substituted heteroaryl group.

“Carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

Particular examples of heterocyclyl groups are shown in the following illustrative examples:

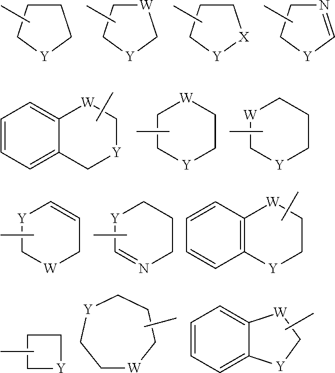

wherein each W is selected from CR⁶⁷, C(R⁶⁷)₂, NR⁶⁷, O, and S; and each Y is selected from NR⁶⁷, O, and S; and R⁶⁷ is independently hydrogen, C₁-C₈ alkyl, C₃-C₁₀ cycloalkyl, 4-10 membered heterocyclyl, C₆-C₁₀ aryl, 5-10 membered heteroaryl. These heterocyclyl rings may be optionally substituted with one or more substituents selected from the group consisting of the group consisting of acyl, acylamino, acyloxy, alkoxy, alkoxycarbonyl, alkoxycarbonylamino, amino, substituted amino, aminocarbonyl (carbamoyl or amido), aminocarbonylamino, aminosulfonyl, sulfonylamino, aryl, aryloxy, azido, carboxyl, cyano, cycloalkyl, halogen, hydroxy, keto, nitro, thiol, —S-alkyl, —S-aryl, —S(O)-alkyl, —S(O)-aryl, —S(O)₂-alkyl, and —S(O)₂-aryl. Substituting groups include carbonyl or thiocarbonyl which provide, for example, lactam and urea derivatives.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl, cycloalkyl, e.g., heterocyclyl, aryl, e.g., heteroaryl, cycloalkenyl, e.g., cycloheteroalkenyl, and the like having from 1 to 5, and particularly from 1 to 3 heteroatoms.

“Acyl” refers to a radical —C(O)R²⁰, where R²⁰ is hydrogen, substituted or unsubstitued alkyl, substituted or unsubstitued alkenyl, substituted or unsubstitued alkynyl, substituted or unsubstitued carbocyclyl, substituted or unsubstituted heterocyclyl, substituted or unsubstituted aryl, or substituted or unsubstitued heteroaryl, as defined herein. “Alkanoyl” is an acyl group wherein R²⁰ is a group other than hydrogen. Representative acyl groups include, but are not limited to, formyl (—CHO), acetyl (—C(═O)CH₃), cyclohexylcarbonyl, cyclohexylmethylcarbonyl, benzoyl (—C(═O)Ph), benzylcarbonyl (—C(═O)CH₂Ph), —C(O)—C₁-C₈ alkyl, —C(O)—(CH₂)ₜ(C₆-C₁₀ aryl), —C(O)—(CH₂)ₜ(5-10 membered heteroaryl), —C(O)—(CH₂)ₜ(C₃-C₁₀ cycloalkyl), and —C(O)—(CH₂)ₜ(4-10 membered heterocyclyl), wherein t is an integer from 0 to 4. In certain embodiments, R²¹ is C₁-C₈ alkyl, substituted with halo or hydroxy; or C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

“Acylamino” refers to a radical —NR²²C(O)R²³, where each instance of R²² and R23 is independently hydrogen, substituted or unsubstitued alkyl, substituted or unsubstitued alkenyl, substituted or unsubstitued alkynyl, substituted or unsubstitued carbocyclyl, substituted or unsubstituted heterocyclyl, substituted or unsubstituted aryl, or substituted or unsubstitued heteroaryl, as defined herein, or R²² is an amino protecting group. Exemplary “acylamino” groups include, but are not limited to, formylamino, acetylamino, cyclohexylcarbonylamino, cyclohexylmethyl-carbonylamino, benzoylamino and benzylcarbonylamino. Particular exemplary “acylamino” groups are —NR²⁴C(O)—C₁-C₈ alkyl, —NR²⁴C(O)—(CH₂)ₜ(C₆-C₁₀ aryl), —NR²⁴C(O)—(CH₂)ₜ(5-10 membered heteroaryl), —NR²⁴C(O)—(CH₂)ₜ(C₃-C₁₀ cycloalkyl), and —NR²⁴C(O)—(CH₂)ₜ(4-10 membered heterocyclyl), wherein t is an integer from 0 to 4, and each R²⁴ independently represents H or C₁-C₈ alkyl. In certain embodiments, R²⁵ is H, C₁-C₈ alkyl, substituted with halo or hydroxy; C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; and R²⁶ is H, C₁-C₈ alkyl, substituted with halo or hydroxy; C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₄-C₄ alkyl, halo, unsubstituted C₄-C₄ alkoxy, unsubstituted C₄-C₄ haloalkyl, unsubstituted C₁-C₄ hydroxyalkyl, or unsubstituted C₄₋C₄ haloalkoxy or hydroxyl; provided that at least one of R²⁵ and R²⁶ is other than H.

“Acyloxy” refers to a radical —OC(O)R²⁷, where R²⁷ is hydrogen, substituted or unsubstitued alkyl, substituted or unsubstitued alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. Representative examples include, but are not limited to, formyl, acetyl, cyclohexylcarbonyl, cyclohexylmethylcarbonyl, benzoyl and benzylcarbonyl. In certain embodiments, R²⁸ is C₁-C₈ alkyl, substituted with halo or hydroxy; C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₄-C₄ alkyl, halo, unsubstituted C₁-C₄ alkoxy, unsubstituted C₁-C₄ haloalkyl, unsubstituted C₄-C₄ hydroxyalkyl, or unsubstituted C₁-C₄ haloalkoxy or hydroxy.

“Alkoxy” refers to the group —OR²⁹ where R²⁹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e. with between 1 and 6 carbon atoms. Further particular alkoxy groups have between 1 and 4 carbon atoms.

In certain embodiments, R²⁹ is a group that has 1 or more substituents, for instance, from 1 to 5 substituents, and particularly from 1 to 3 substituents, in particular 1 substituent, selected from the group consisting of amino, substituted amino, C₆-C₁₀ aryl, aryloxy, carboxyl, cyano, C₃-C₁₀ cycloalkyl, 4-10 membered heterocyclyl, halogen, 5-10 membered heteroaryl, hydroxyl, nitro, thioalkoxy, thioaryloxy, thiol, alkyl-S(O)—, aryl-S(O)—, alkyl-S(O)₂— and aryl-S(O)₂—. Exemplary ‘substituted alkoxy’ groups include, but are not limited to, —O—(CH₂)ₜ(C₆-C₁₀ aryl), —O—(CH₂)ₜ(5-10 membered heteroaryl), —O—(CH₂)ₜ(C₃-C₁₀ cycloalkyl), and —O—(CH₂)ₜ(4-10 membered heterocyclyl), wherein t is an integer from 0 to 4 and any aryl, heteroaryl, cycloalkyl or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Particular exemplary ‘substituted alkoxy’ groups are —OCF₃, —OCH₂CF₃, —OCH₂Ph, —OCH₂-cyclopropyl, —OCH₂CH₂OH, and —OCH₂CH₂NMe₂.

“Amino” refers to the radical —NH₂.

“Substituted amino” refers to an amino group of the formula —N(R³⁸)₂ wherein R³⁸ is hydrogen, substituted or unsubstituted alkyl, substituted or unsubstitued alkenyl, substituted or unsubstitued alkynyl, substituted or unsubstitued carbocyclyl, substituted or unsubstituted heterocyclyl, substituted or unsubstituted aryl, substituted or unsubstitued heteroaryl, or an amino protecting group, wherein at least one of R³⁸ is not a hydrogen. In certain embodiments, each R³⁸ is independently selected from: hydrogen, C₄-C₈ alkyl, C₃-C₈ alkenyl, C₃-C₈ alkynyl, C₆-C₁₀ aryl, 5-10 membered heteroaryl, 4-10 membered heterocyclyl, or C₃-C₁₀ cycloalkyl; or C₁-C₈ alkyl, substituted with halo or hydroxy; C₃-C₈ alkenyl, substituted with halo or hydroxy; C₃-C₈ alkynyl, substituted with halo or hydroxy, or —(CH₂)ₜ(C₆-C₁₀ aryl), —(CH₂)ₜ(5-10 membered heteroaryl), —(CH₂)ₜ(C₃-C₁₀ cycloalkyl), or —(CH₂)ₜ(4-10 membered heterocyclyl), wherein t is an integer between 0 and 8, each of which is substituted by unsubstituted C₄-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; or both R³⁸ groups are joined to form an alkylene group.

Exemplary ‘substituted amino’ groups are —NR³⁹—C₁-C₈ alkyl, —NR³⁹—(CH₂)ₜ(C₆-C₁₀ aryl), —NR³⁹—(CH₂)ₜ(5-10 membered heteroaryl), —NR³⁹—(CH₂)ₜ(C₃-C₁₀ cycloalkyl), and —NR³⁹—(CH₂)ₜ(4-10 membered heterocyclyl), wherein t is an integer from 0 to 4, for instance 1 or 2, each R³⁹ independently represents H or C₁-C₈ alkyl and any alkyl groups present, may themselves be substituted by halo, substituted or unsubstituted amino, or hydroxy; and any aryl, heteroaryl, cycloalkyl, or heterocyclyl groups present, may themselves be substituted by unsubstituted C₄-C₄ alkyl, halo, unsubstituted C₁-C₄ alkoxy, unsubstituted C₁-C₄ haloalkyl, unsubstituted C₄-C₄ hydroxyalkyl, or unsubstituted C₁-C₄ haloalkoxy or hydroxy. For the avoidance of doubt the term ‘substituted amino’ includes the groups alkylamino, substituted alkylamino, alkylarylamino, substituted alkylarylamino, arylamino, substituted arylamino, dialkylamino, and substituted dialkylamino as defined below. Substituted amino encompasses both monosubstituted amino and disubstituted amino groups.

“Azido” refers to the radical —N₃.

“Carbamoyl” or “amido” refers to the radical —C(O)NH₂.

“Substituted carbamoyl” or “substituted amido” refers to the radical —C(O)N(R⁶²)₂ wherein each R⁶² is independently hydrogen, substituted or unsubstituted alkyl, substituted or unsubstitued alkenyl, substituted or unsubstitued alkynyl, substituted or unsubstitued carbocyclyl, substituted or unsubstituted heterocyclyl, substituted or unsubstituted aryl, substituted or unsubstitued heteroaryl, or an amino protecting group, wherein at least one of R⁶² is not a hydrogen. In certain embodiments, R⁶² is selected from H, C₁-C₈ alkyl, C₃-C₁₀ cycloalkyl, 4-10 membered heterocyclyl, C₆-C₁₀ aryl, aralkyl, 5-10 membered heteroaryl, and heteroaralkyl; or C₁-C₈ alkyl substituted with halo or hydroxy; or C₃-C₁₀ cycloalkyl, 4-10 membered heterocyclyl, C₆-C₁₀ aryl, aralkyl, 5-10 membered heteroaryl, or heteroaralkyl, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; provided that at least one R⁶² is other than H.

Exemplary ‘substituted carbamoyl’ groups include, but are not limited to, —C(O) NR⁶⁴—C₁-C₈ alkyl, —C(O)NR⁶⁴—(CH₂)ₜ(C₆-C₁₀ aryl). —C(O)N⁶⁴—(CH₂)ₜ(5-10 membered heteroaryl), —C(O)NR⁶⁴—(CH₂)ₜ(C₃-C₁₀ cycloalkyl), and —C(O)NR⁶⁴—(CH₂)ₜ(4-10 membered heterocyclyl), wherein t is an integer from 0 to 4, each R⁶⁴ independently represents H or C₁-C₈ alkyl and any aryl, heteroaryl, cycloalkyl or heterocyclyl groups present, may themselves be substituted by unsubstituted C₄-C₄ alkyl, halo, unsubstituted C₁-C₄ alkoxy, unsubstituted C₁-C₄ haloalkyl, unsubstituted C₄-C₄ hydroxyalkyl, or unsubstituted C₁-C₄ haloalkoxy or hydroxy.

‘Carboxy’ refers to the radical —C(O)OH.

“Cyano” refers to the radical —CN.

“Halo” or “halogen” refers to fluoro (F), chloro (Cl), bromo (Br), and iodo (I). In certain embodiments, the halo group is either fluoro or chloro. In further embodiments, the halo group is iodo.

“Hydroxy” refers to the radical —OH.

“Nitro” refers to the radical —NO₂.

“Cycloalkylalkyl” refers to an alkyl radical in which the alkyl group is substituted with a cycloalkyl group. Typical cycloalkylalkyl groups include, but are not limited to, cyclopropylmethyl, cyclobutylmethyl, cyclopentylmethyl, cyclohexylmethyl, cycloheptylmethyl, cyclooctylmethyl, cyclopropylethyl, cyclobutylethyl, cyclopentylethyl, cyclohexylethyl, cycloheptylethyl, and cyclooctylethyl, and the like.

“Heterocyclylalkyl” refers to an alkyl radical in which the alkyl group is substituted with a heterocyclyl group. Typical heterocyclylalkyl groups include, but are not limited to, pyrrolidinylmethyl, piperidinylmethyl, piperazinylmethyl, morpholinylmethyl, pyrrolidinylethyl, piperidinylethyl, piperazinylethyl, morpholinylethyl, and the like.

“Cycloalkenyl” refers to substituted or unsubstituted carbocyclyl group having from 3 to 10 carbon atoms and having a single cyclic ring or multiple condensed rings, including fused and bridged ring systems and having at least one and particularly from 1 to 2 sites of olefinic unsaturation. Such cycloalkenyl groups include, by way of example, single ring structures such as cyclohexenyl, cyclopentenyl, cyclopropenyl, and the like.

“Fused cycloalkenyl” refers to a cycloalkenyl having two of its ring carbon atoms in common with a second aliphatic or aromatic ring and having its olefinic unsaturation located to impart aromaticity to the cycloalkenyl ring.

“Ethenyl” refers to substituted or unsubstituted —(C═C)—.

“Ethylene” refers to substituted or unsubstituted —(C—C)—.

“Ethynyl” refers to —(C═C)—.

“Nitrogen-containing heterocyclyl” group means a 4- to 7-membered non-aromatic cyclic group containing at least one nitrogen atom, for example, but without limitation, morpholine, piperidine (e.g. 2-piperidinyl, 3-piperidinyl and 4-piperidinyl), pyrrolidine (e.g. 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

“Thioketo” refers to the group ═S.

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc}; each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C_{W} alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC_{W} alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X is a counterion.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is a nitrogen protecting group (also referred to as an amino protecting group). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, A-benzoylphenylalanyl derivative, benzamide, p-phenylbenzamide, o-nitophenylacetamide, o-nitrophenoxy acetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on an sulfur atom is an sulfur protecting group (also referred to as a thiol protecting group). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

“Compounds of the present invention”, and equivalent expressions, are meant to embrace the compounds as hereinbefore described, in particular compounds according to any of the Formula herein recited and/or described, which expression includes the prodrugs, the pharmaceutically acceptable salts, and the solvates, e.g., hydrates, where the context so permits. Similarly, reference to intermediates, whether or not they themselves are claimed, is meant to embrace their salts, and solvates, where the context so permits.

These and other exemplary substituents are described in more detail in the Detailed Description, Examples, and claims. The invention is not intended to be limited in any manner by the above exemplary listing of substituents.

### Other Definitions

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like (see, e.g., Berge, et al., J. Pharm. Sci. 66(1): 1-79 (January '77).

“Pharmaceutically acceptable vehicle” refers to a diluent, adjuvant, excipient or carrier with which a compound of the invention is administered.

“Pharmaceutically acceptable metabolically cleavable group” refers to a group which is cleaved in vivo to yield the parent molecule of the structural Formula indicated herein. Examples of metabolically cleavable groups include —COR, —COOR, —CONRR and —CH₂OR radicals, where R is selected independently at each occurrence from alkyl, trialkylsilyl, carbocyclic aryl or carbocyclic aryl substituted with one or more of alkyl, halogen, hydroxy or alkoxy. Specific examples of representative metabolically cleavable groups include acetyl, methoxycarbonyl, benzoyl, methoxymethyl and trimethylsilyl groups.

“Prodrugs” refers to compounds, including derivatives of the compounds of the invention, which have cleavable groups and become by solvolysis or under physiological conditions the compounds of the invention that are pharmaceutically active in vivo. Such examples include, but are not limited to, choline ester derivatives and the like, N-alkylmorpholine esters and the like. Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are particular prodrugs. In some cases it is desirable to prepare double ester type prodrugs such as (acyloxy)alkyl esters or ((alkoxycarbonyl)oxy)alkylesters. Particularly the C₁ to C₈ alkyl, C₂-C₈ alkenyl, C₂-C₈ alkynyl, aryl, C₇-C₁₂ substituted aryl, and C₇-C₁₂ arylalkyl esters of the compounds of the invention.

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human”, “patient” and “subject” are used interchangeably herein.

“Therapeutically effective amount” means the amount of a compound that, when administered to a subject for treating a disease, is sufficient to effect such treatment for the disease. The “therapeutically effective amount” can vary depending on the compound, the disease and its severity, and the age, weight, etc., of the subject to be treated.

“Preventing” or “prevention” refers to a reduction in risk of acquiring or developing a disease or disorder (i.e., causing at least one of the clinical symptoms of the disease not to develop in a subject not yet exposed to a disease-causing agent, or predisposed to the disease in advance of disease onset.

The term “prophylaxis” is related to “prevention”, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

“Treating” or “treatment” of any disease or disorder refers, in certain embodiments, to ameliorating the disease or disorder (i.e., arresting the disease or reducing the manifestation, extent or severity of at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

As used herein, the term “isotopic variant” refers to a compound that contains unnatural proportions of isotopes at one or more of the atoms that constitute such compound. For example, an “isotopic variant” of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e., ³H, and carbon-14, i.e., ¹⁴C, are particularly useful for this purpose in view of their ease of incorporation and ready means of detection. Further, compounds may be prepared that are substituted with positron emitting isotopes, such as ¹¹C, ¹⁸F, ¹⁵O and ¹³N, and would be useful in Positron Emission Topography (PET) studies for examining substrate receptor occupancy. All isotopic variants of the compounds provided herein, radioactive or not, are intended to be encompassed within the scope of the invention.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, when it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of t electrons and an atom (usually H). For example, enols and ketones are tautomers because they are rapidly interconverted by treatment with either acid or base. Another example of tautomerism is the aci- and nitro-forms of phenylnitromethane, which are likewise formed by treatment with acid or base. Tautomeric forms may be relevant to the attainment of the optimal chemical reactivity and biological activity of a compound of interest.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure R-compound” refers to at least about 80% by weight R-compound and at most about 20% by weight S-compound, at least about 90% by weight R-compound and at most about 10% by weight S-compound, at least about 95% by weight R-compound and at most about 5% by weight S-compound, at least about 99% by weight R-compound and at most about 1% by weight S-compound, at least about 99.9% by weight R-compound or at most about 0.1% by weight S-compound. In certain embodiments, the weights are based upon total weight of compound.

As used herein and unless otherwise indicated, the term “enantiomerically pure S-compound” or “S-compound” refers to at least about 80% by weight S-compound and at most about 20% by weight R-compound, at least about 90% by weight S-compound and at most about 10% by weight R-compound, at least about 95% by weight S-compound and at most about 5% by weight R-compound, at least about 99% by weight S-compound and at most about 1% by weight R-compound or at least about 99.9% by weight S-compound and at most about 0.1% by weight R-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

The compounds of this invention may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

One having ordinary skill in the art of organic synthesis will recognize that the maximum number of heteroatoms in a stable, chemically feasible heterocyclic ring, whether it is aromatic or non aromatic, is determined by the size of the ring, the degree of unsaturation and the valence of the heteroatoms. In general, a heterocyclic ring may have one to four heteroatoms so long as the heteroaromatic ring is chemically feasible and stable.

### DETAILED DESCRIPTION OF THE PREFERRED EMBODIMENTS

In certain aspects, provided herein are pharmaceutical compositions comprising a bolaamphiphile vesicle complex.

In certain aspects, the bolaamphiphile vesicle complexes comprise one or more bolaamphiphilic compounds and a biologically active compound. In a particular embodiment, the biologically active compound is siRNA.

In further aspects, provided herein are novel siRNA and bolamphiphilic complex comprising siRNA and one or more bolaamphiphilic compounds.

In further aspects, provided herein are novel formulations of siRNA with bolaamphiphilic compounds or with bolaamphiphile vesicles.

In another aspect, provided here are methods of delivering siRNA into animal or human cell comprising the step of administering to the animal or human a pharmaceutical composition comprising a bolaamphiphile vesicle complex; and wherein the bolaamphiphile vesicle complex comprises one or more bolaamphiphilic compounds and siRNA. In one embodiment, the cell is brain cell, liver cell, gall bladder cell, or a lung cell. In other embodiments, the cells are are cells of a lymph node, a CD4+ lymphocyte, or a cell of the mononuclear phagocyte system, including, without limitation, a monocyte, macrophage, a resident brain microglial cell and a dendritic cell.

In another aspect, provided here are methods of delivering siRNA into animal or human organs comprising the step of administering to the animal or human a pharmaceutical composition comprising of a bolaamphiphile vesicle complex; and wherein the bolaamphiphile vesicle complex comprises one or more bolaamphiphilic compounds and siRNA. In one embodiment, the organ is brain, liver, gall bladder, or a lung.

In one embodiment, the bolaamphiphilic complex comprises one bolaamphiphilic compound. In another embodiment, the bolaamphiphilic complex comprises two bolaamphiphilic compounds.

In one embodiment, the bolaamphiphilic compound consists of two hydrophilic headgroups linked through a long hydrophobic chain. In another embodiment, the hydrophilic headgroup is an amino containing group. In a specific embodiment, the hydrophilic headgroup is a tertiary or quaternary amino containing group.

In one particular embodiment, the bolaamphiphilic compound is a compound according to formula I:

HG²-L¹-HG¹ I

or a pharmaceutically acceptable salt, solvate, hydrate, prodrug, stereoisomer, tautomer, isotopic variant, or N-oxide thereof, or a combination thereof;
wherein:

each HG¹ and HG² is independently a hydrophilic head group; and

L¹ is alkylene, alkenyl, heteroalkylene, or heteroalkenyl linker; unsubstituted or substituted with C₁-C₂₀ alkyl, hydroxyl, or oxo.

In one embodiment, the pharmaceutically acceptable salt is a quaternary ammonium salt.

In one embodiment, with respect to the bolaamphiphilic compound of formula I, L¹ is heteroalkylene, or heteroalkenyl linker comprising C, N, and O atoms; unsubstituted or substituted with C₁-C₂₀ alkyl, hydroxyl, or oxo.

In another embodiment, with respect to the bolaamphiphilic compound of formula I, L¹ is

—O-L²-C(O)—O—(CH₂)ₙ₄—O—C(O)-L³-O—, or

—O-L²-C(O)—O—(CH₂)ₙ₅—O—C(O)—(CH₂)ₙ₆—,
and wherein each L² and L³ is C₄-C₂₀ alkenyl linker; unsubstituted or substituted with C₁-C₈ alkyl or hydroxy;
and n4, n5, and n6 is independently an integer from 4-20.

In one embodiment, each L² and L³ is independently —C(R¹)—C(OH)—CH₂—(CH═CH)—(CH₂)ₙ₇—; R¹ is C₁-C₈ alkyl, and n7 is independently an integer from 4-20.

In another embodiment, with respect to the bolaamphiphilic compound of formula I, L¹ is —O—(CH₂)ₙ₁—O—C(O)—(CH₂)ₙ₂—C(O)—O—(CH₂)ₙ₃—O—.

In another embodiment, with respect to the bolaamphiphilic compound of formula I, L¹ is

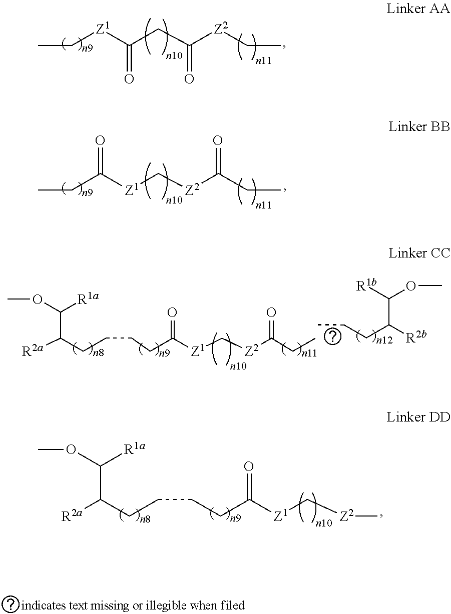

wherein:
each Z¹ and Z² is independently —C(R³)₂—, —N(R³)— or —O—;
each R^{1a}, R^{1b}, R³, and R⁴ is independently H or C₁-C₈ alkyl;
each R^{2a} and R^{2b} is independently H, C₁-C₈ alkyl, OH, or alkoxy;
each n8, n9, n11, and n12 is independently an integer from 1-20;
n10 is an integer from 2-20; and
each dotted bond is independently a single or a double bond,
and wherein each methylene carbon is unsubstituted or substituted with C₁-C₄ alkyl; and each n1, n2, and n3 is independently an integer from 4-20.

In one embodiment, with respect to the bolaamphiphilic compound of formula I, the bolaamphiphilic compound is a compound according to formula II, III, IV, V, or VI:

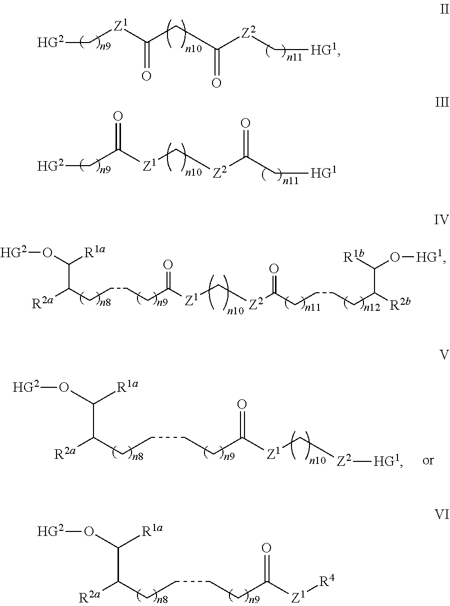

or a pharmaceutically acceptable salt, solvate, hydrate, prodrug, stereoisomer, tautomer, isotopic variant, or N-oxide thereof, or a combination thereof;
wherein:
each HG¹ and HG² is independently a hydrophilic head group;
each Z¹ and Z² is independently —C(R³)₂—, —N(R³)— or —O—;
each R^{1a}, R^{1b}, R³, and R⁴ is independently H or C₁-C₈ alkyl;
each R^{2a} and R^{2b} is independently H, C₁-C₈ alkyl, OH, alkoxy, or O-HG¹ or O-HG²;
each n8, n9, n11, and n12 is independently an integer from 1-20;
n10 is an integer from 2-20; and
each dotted bond is independently a single or a double bond.

In one embodiment, with respect to the bolaamphiphilic compound of formula II, III, IV, V, or VI, each n9 and n11 is independently an integer from 2-12. In another embodiment, n9 and n11 is independently an integer from 4-8. In a particular embodiment, each n9 and n11 is 7 or 11.

In one embodiment, with respect to the bolaamphiphilic compound of formula II, III, IV, V, or VI, each n8 and n12 is independently 1, 2, 3, or 4. In a particular embodiment, each n8 and n12 is 1.

In one embodiment, with respect to the bolaamphiphilic compound of formula II, III, IV, V, or VI, each R^{2a} and R^{2b} is independently H, OH, or alkoxy. In another embodiment, each R^{2a} and R^{2b} is independently H, OH, or OMe. In another embodiment, each R^{2a} and R^{2b} is independently-O-HG¹ or O-HG². In a particular embodiment, each R^{2a} and R^{2b} is OH.

In one embodiment, with respect to the bolaamphiphilic compound of formula II, III, IV, V, or VI, each R^{1a} and R^{1b} is independently H, Me, Et, n-Pr, i-Pr, n-Bu, i-Bu, sec-Bu, n-pentyl, isopentyl, n-hexyl, n-heptyl, or n-octyl. In a particular embodiment, each R^{1a} and R^{1b} is independently n-pentyl.

In one embodiment, with respect to the bolaamphiphilic compound of formula II, III, IV, V, or VI, each dotted bond is a single bond. In another embodiment, each dotted bond is a double bond.

In one embodiment, with respect to the bolaamphiphilic compound of formula II, III, IV, V, or VI, n10 is an integer from 2-16. In another embodiment, n10 is an integer from 2-12. In a particular embodiment, n10 is 2, 4, 6, 8, 10, 12, or 16.

In one embodiment, with respect to the bolaamphiphilic compound of formula IV, R⁴ is H, Me, Et, n-Pr, i-Pr, n-Bu, i-Bu, sec-Bu, n-pentyl, or isopentyl. In another embodiment, R⁴ is Me, or Et. In a particular embodiment, R⁴ is Me.

In one embodiment, with respect to the bolaamphiphilic compound of formula II, III, IV, V, or VI, each Z¹ and Z² is independently C(R³)₂—, or —N(R³)—. In another embodiment, each Z¹ and Z² is independently C(R³)₂—, or —N(R³)—; and each R³ is independently H, Me, Et, n-Pr, i-Pr, n-Bu, i-Bu, sec-Bu, n-pentyl, or isopentyl. In a particular embodiment, R³ is H.

In one embodiment, with respect to the bolaamphiphilic compound of formula II, III, IV, V, or VI, each Z¹ and Z² is —O—.

In one embodiment, with respect to the bolaamphiphilic compound of formula I, II, III, or IV, each HG¹ and HG² is independently selected from:

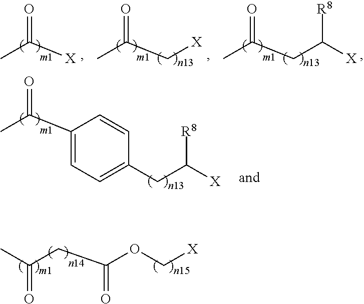

wherein:
X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocyclyl;
each R^{5c} is independently substituted or unsubstituted C₁-C₂₀ alkyl; each R⁸ is independently H, substituted or unsubstituted C₁-C₂₀ alkyl, alkoxy, or carboxy;
m1 is 0 or 1; and
each n13, n14, and n15 is independently an integer from 1-20.

In one embodiment, with respect to the bolaamphiphilic compound of formula I, II, III, or IV, HG¹ and HG² are as defined above, and each m1 is 0.

In one embodiment, with respect to the bolaamphiphilic compound of formula I, II, III, or IV, HG¹ and HG² are as defined above, and each m1 is 1.

In one embodiment, with respect to the bolaamphiphilic compound of formula I, II, III, or IV, HG¹ and HG² are as defined above, and each n13 is 1 or 2.

In one embodiment, with respect to the bolaamphiphilic compound of formula I, II, III, or IV, HG¹ and HG² are as defined above, and each n14 and n15 is independently 1, 2, 3, 4, or 5. In another embodiment, each n14 and n15 is independently 2 or 3.

In one particular embodiment, the bolaamphiphilic compound is a compound according to formula VIIa, VIIb, VIIc, or VIId:

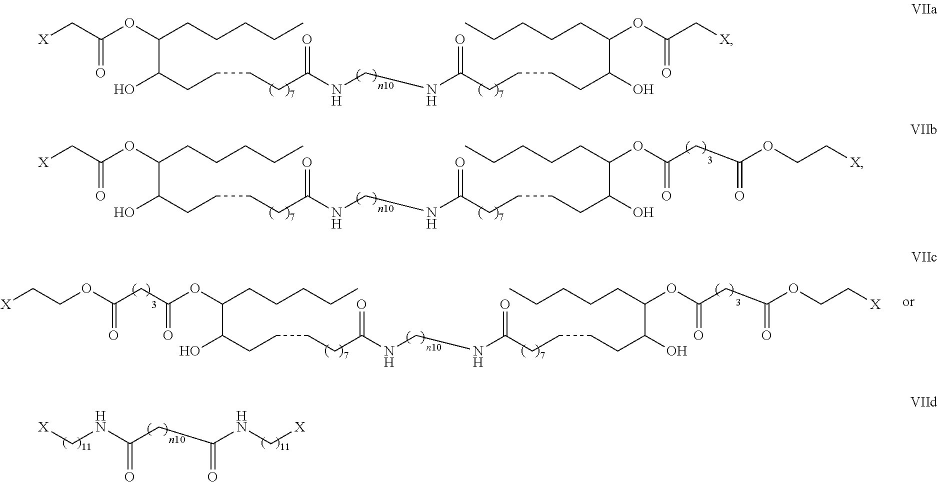

or a pharmaceutically acceptable salt, solvate, hydrate, prodrug, stereoisomer, tautomer, isotopic variant, or N-oxide thereof, or a combination thereof;
wherein:
each X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocyclyl;
   each R^{5c} is independently substituted or unsubstituted C₁-C₂₀ alkyl;
   n10 is an integer from 2-20; and
   each dotted bond is independently a single or a double bond.

In another particular embodiment, the bolaamphiphilic compound is a compound according to formula VIIIa, VIIIb, VIIIc, or VIIId:

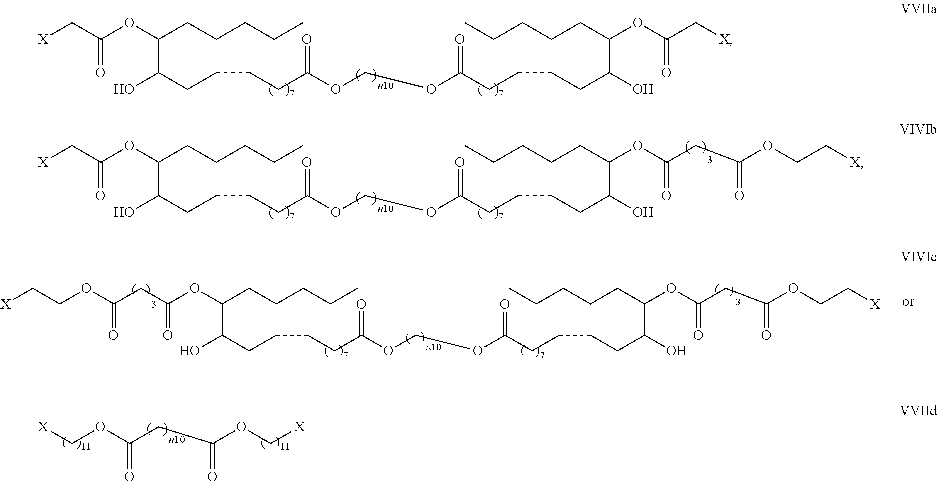

or a pharmaceutically acceptable salt, solvate, hydrate, prodrug, stereoisomer, tautomer, isotopic variant, or N-oxide thereof, or a combination thereof;
wherein:
each X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocyclyl;
   each R^{5c} is independently substituted or unsubstituted C₁-C₂₀ alkyl;
   n10 is an integer from 2-20; and
   each dotted bond is independently a single or a double bond.

In another particular embodiment, the bolaamphiphilic compound is a compound according to formula IXa, IXb, or IXc:

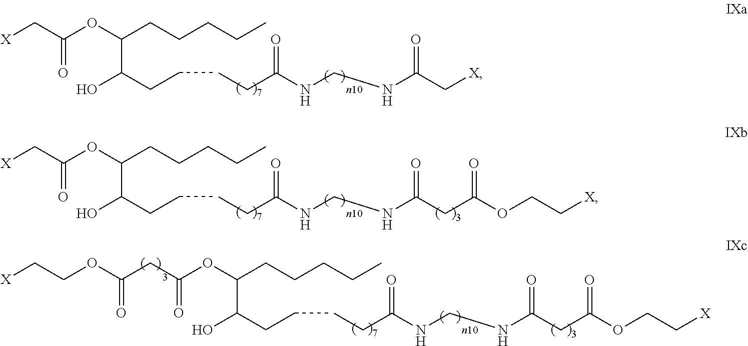

or a pharmaceutically acceptable salt, solvate, hydrate, prodrug, stereoisomer, tautomer, isotopic variant, or N-oxide thereof, or a combination thereof;
wherein:
each X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocyclyl;
   each R^{5c} is independently substituted or unsubstituted C₁-C₂₀ alkyl;
   n10 is an integer from 2-20; and
   each dotted bond is independently a single or a double bond.

In another particular embodiment, the bolaamphiphilic compound is a compound according to formula Xa, Xb, or Xc:

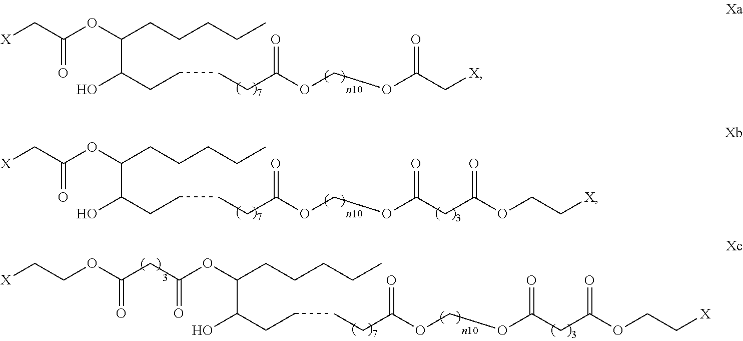

or a pharmaceutically acceptable salt, solvate, hydrate, prodrug, stereoisomer, tautomer, isotopic variant, or N-oxide thereof, or a combination thereof;
wherein:
each X is —NR^{5a}R^{5b}, or —N⁺R^{5a}R^{5b}R^{5c}; each R^{5a}, and R^{5b} is independently H or substituted or unsubstituted C₁-C₁₀ alkyl or R^{5a} and R^{5b} may join together to form an N containing substituted or unsubstituted heteroaryl, or substituted or unsubstituted heterocyclyl;
   each R^{5c} is independently substituted or unsubstituted C₁-C₁₀ alkyl;
   n10 is an integer from 2-20; and
   each dotted bond is independently a single or a double bond.

In one embodiment, with respect to the bolaamphiphilic compound of formula VIIa-VIId, VIIIa-VIIId, IXa-IXc, or Xa-Xc, each dotted bond is a single bond. In another embodiment, each dotted bond is a double bond.

In one embodiment, with respect to the bolaamphiphilic compound of formula VIIa-VIId, VIIIa-VIIId, IXa-IXc, or Xa-Xc, n10 is an integer from 2-16.

In one embodiment, with respect to the bolaamphiphilic compound of formula VIIa-VIId, VIIIa-VIIId, IXa-IXc, or Xa-Xc, n10 is an integer from 2-12.

In one embodiment, with respect to the bolaamphiphilic compound of formula VIIa-VIId, VIIIa-VIIId, IXa-IXc, or Xa-Xc, n10 is 2, 4, 6, 8, 10, 12, or 16.

In one embodiment, with respect to the bolaamphiphilic compound of formula VIIa-VIId, VIIIa-VIIId, IXa-IXc, or Xa-Xc, each R^{5a}, R^{5b}, and R^{5c} is independently substituted or unsubstituted C₁-C₂₀ alkyl.

In one embodiment, with respect to the bolaamphiphilic compound of formula VIIa-VIId, VIIIa-VIIId, IXa-IXc, or Xa-Xc, each R^{5a}, R^{5b}, and R^{5c} is independently unsubstituted C₁-C₂₀ alkyl.

In one embodiment, with respect to the bolaamphiphilic compound of formula VIIa-VIId, VIIIa-VIIId, IXa-IXc, or Xa-Xc, one of R^{5a}, R^{5b}, and R^{5c} is C₁-C₂₀ alkyl substituted with —OC(O)R⁶; and R⁶ is C₁-C₂₀ alkyl.

In one embodiment, with respect to the bolaamphiphilic compound of formula VIIa-VIId, VIIIa-VIIId, IXa-IXc, or Xa-Xc, two of R^{5a}, R^{5b}, and R^{5c} are independently C₁-C₂₀ alkyl substituted with —OC(O)R⁶; and R⁶ is C₁-C₂₀ alkyl. In one embodiment, R⁶ is Me, Et, n-Pr, i-Pr, n-Bu, i-Bu, sec-Bu, n-pentyl, isopentyl, n-hexyl, n-heptyl, or n-octyl. In a particular embodiment, R⁶ is Me.

In one embodiment, with respect to the bolaamphiphilic compound of formula VIIa-VIId, VIIIa-VIIId, IXa-IXc, or Xa-Xc, one of R^{5a}, R^{5b}, and R^{5c} is C₁-C₂₀ alkyl substituted with amino, alkylamino or dialkylamino.

In one embodiment, with respect to the bolaamphiphilic compound of formula VIIa-VIId, VIIIa-VIIId, IXa-IXc, or Xa-Xc, two of R^{5a}, R^{5b}, and R^{5c} are independently C₁-C₂₀ alkyl substituted with amino, alkylamino or dialkylamino.

In one embodiment, with respect to the bolaamphiphilic compound of formula VIIa-VIId, VIIIa-VIIId, IXa-IXc, or Xa-Xc, R^{5a}, and R^{5b} together with the N they are attached to form substituted or unsubstituted heteroaryl.

In one embodiment, with respect to the bolaamphiphilic compound of formula VIIa-VIId, VIIIa-VIIId, IXa-IXc, or Xa-Xc, R^{5a}, and R^{5b} together with the N they are attached to form substituted or unsubstituted pyridyl.

In one embodiment, with respect to the bolaamphiphilic compound of formula VIIa-VIId, VIIIa-VIIId, IXa-IXc, or Xa-Xc, R^{5a}, and R^{5b} together with the N they are attached to form substituted or unsubstituted monocyclic or bicyclic heterocyclyl.

In one embodiment, with respect to the bolaamphiphilic compound of formula VIIa-VIId, VIIIa-VIIId, IXa-IXc, or Xa-Xc, X is substituted or unsubstituted

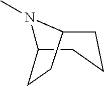

In one embodiment, with respect to the bolaamphiphilic compound of formula VIIa-VIId, VIIIa-VIIId, IXa-IXc, or Xa-Xc, X is

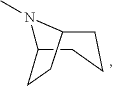

substituted with one or more groups selected from alkoxy, acetyl, and substituted or unsubstituted Pb.

In one embodiment, with respect to the bolaamphiphilic compound of formula VIIa-VIId, VIIIa-VIIId, IXa-IXc, or Xa-Xc, X is

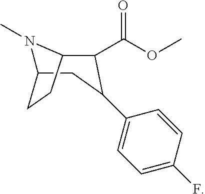 [SMILES: COC(=O)C1C(c2ccc(F)cc2)CC2CCC1N2C]

In one embodiment, with respect to the bolaamphiphilic compound of formula VIIa-VIId, VIIIa-VIIId, IXa-IXc, or Xa-Xc, X is —NMe₂ or —N⁺Me₃.

In one embodiment, with respect to the bolaamphiphilic compound of formula VIIa-VIId, VIIIa-VIIId, IXa-IXc, or Xa-Xc, X is —N(Me)-CH₂CH₂—OAc or —N⁺(Me)₂-CH₂CH₂—OAc.

In one embodiment, with respect to the bolaamphiphilic compound of formula VIIa-VIId, VIIIa-VIIId, IXa-IXc, or Xa-Xc, X is a chitosanyl group; and the chitosanyl group is a poly-(D)glucosaminyl group with MW of 3800 to 20,000 Daltons, and is attached to the core via N.

In one embodiment, the chitosanyl group is

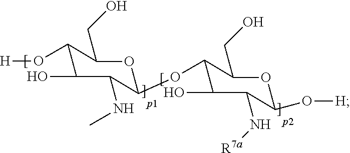

and wherein each p1 and p2 is independently an integer from 1-400; and each R^{7a} is H or acyl.

In one embodiment, with respect to the bolaamphiphilic compound of formula I, II, III, IV, V, VI, VIIa-VIIc, VIIIa-VIIIc, IXa-IXc and Xa-Xc, the bolaamphiphilic compound is a pharmaceutically acceptable salt.

In one embodiment, with respect to the bolaamphiphilic compound of formula I, II, III, IV, V, VI, VIIa-VIIc, VIIIa-VIIIc, IXa-IXc and Xa-Xc, the bolaamphiphilic compound is in a form of a quaternary salt.

In one embodiment, with respect to the bolaamphiphilic compound of formula I, II, III, IV, V, VI, VIIa-VIIc, VIIIa-VIIIc, IXa-IXc and Xa-Xc, the bolaamphiphilic compound is in a form of a quaternary salt with pharmaceutically acceptable alkyl halide or alkyl tosylate.

In one embodiment, with respect to the bolaamphiphilic compound of formula I, II, III, IV, V, VI, VIIa-VIIc, VIIIa-VIIIc, IXa-IXc and Xa-Xc, the bolaamphiphilic compound is any one of the bolaambphilic compounds listed in Table 1.

In one embodiment, with respect to the bolaamphiphilic compound of formula I, II, III, IV, V, VI, VIIa-VIIc, VIIIa-VIIIc, IXa-IXc and Xa-Xc, the bolaamphiphilic compound is GLH-19.

In one embodiment, with respect to the bolaamphiphilic compound of formula I, II, III, IV, V, VI, VIIa-VIIc, VIIIa-VIIIc, IXa-IXc and Xa-Xc, the bolaamphiphilic compound is GLH-20.

In one embodiment, with respect to the bolaamphiphilic compound of formula I, II, III, IV, V, VI, VIIa-VIIc, VIIIa-VIIIc, IXa-IXc and Xa-Xc, the bolaamphiphilic compound is GLH-16.

In one embodiment, with respect to the bolaamphiphilic compound of formula I, II, III, IV, V, VI, VIIa-VIIc, VIIIa-VIIIc, IXa-IXc and Xa-Xc, the bolaamphiphilic compound is GLH-26, 29, or 41.

In one embodiment, with respect to the bolaamphiphilic compound of formula I, II, III, IV, V, VI, VIIa-VIIc, VIIIa-VIIIc, IXa-IXc and Xa-Xc, the bolaamphiphilic compound is other than Comound ID GLH-16, GLH-19, GLH-20, GLH-26, GLH-29, or GLH-41.

In one embodiment, with respect to the bolaamphiphilic compound of formula I, II, III, IV, V, VI, VIIa-VIIc, VIIIa-VIIIc, IXa-IXc and Xa-Xc, the bolaamphiphilic compound is other than Comound ID GLH-6, GLH-8, GLH-12, GLH-13, GLH-13a, or GLH-49 to GLH-54 (all can be used as intermediates for bolaamphiphiles).

In another specific aspect, provided herein are composition of novel bolaamphiphilic compounds, wherein the bolaamphiphilic compound is selected from the bolaambphilic compounds listed in Table 1. In one embodiment, with respect to the bolaamphiphilic compound, the bolaamphiphilic compound is other than Comound ID GLH-16, GLH-19, GLH-20, GLH-26, GLH-29, or GLH-41. In another embodiment, with respect to the bolaamphiphilic compound, the compound is other than compound with ID GLH-3, GLH-4, GLH-5, or GLH-21.

In one particular embodiment, bolaamphiphilic compound is selected from the bolaambphilic compounds listed in Table 1, and the compound is compound with ID GLH-7, GLH-9, GLH-10, GLH-11, GLH-14, GLH-15, GLH-17, GLH-18, GLH-22, GLH-23, GLH-24, GLH-25, GLH-27, GLH-28, GLH-30 to GLH-48, GLH-55, GLH-56, or GLH-57.

In another specific aspect, provided herein are methods for delivering siRNA across the cell membrane. In one embodiment, the cell is brain cell, liver cell, gall bladder cell, or a lung cell. In other specific aspects, the cells are are cells of a lymph node, a CD4+ lymphocyte, or a cell of the mononuclear phagocyte system, including, without limitation, a monocyte, macrophage, a resident brain microglial cell and a dendritic cell.

In another aspect, provided here are methods of delivering siRNA into animal or human brain comprising the step of administering to the animal or human a pharmaceutical composition comprising of a bolaamphiphile vesicle complex; and wherein the bolaamphiphile vesicle complex comprises one or more bolaamphiphilic compounds and siRNA.

In another aspect, provided here are methods of delivering siRNA into animal or human liver comprising the step of administering to the animal or human a pharmaceutical composition comprising of a bolaamphiphile vesicle complex; and wherein the bolaamphiphile vesicle complex comprises one or more bolaamphiphilic compounds and siRNA.

In another aspect, provided here are methods of delivering siRNA into animal or human lungs comprising the step of administering to the animal or human a pharmaceutical composition comprising of a bolaamphiphile vesicle complex; and wherein the bolaamphiphile vesicle complex comprises one or more bolaamphiphilic compounds and siRNA.

In another aspect, provided here are methods of delivering siRNA into animal or human gall bladder comprising the step of administering to the animal or human a pharmaceutical composition comprising of a bolaamphiphile vesicle complex; and wherein the bolaamphiphile vesicle complex comprises one or more bolaamphiphilic compounds and siRNA.

In another specific aspect, provided herein are nano-particles, comprising one or more bolaamphiphilic compounds and siRNA. In one embodiment, the bolaamphiphilic compounds and siRNA are encapsulated within the nano-particle.

In particular embodiments, polynucleotides selected from DNA or RNA fragments are delivered by the nanoparticles of the invention. In a more particular embodiment, the polynucleotide is a small interfering RNA (siRNA), a double-stranded RNA molecule of 20-25 nucleotides. siRNAs play a variety of roles in biology. Most notably, siRNAs are involved in the RNA interference (RNAi) pathway, where they interfere with the expression of a specific gene. In addition to their role in the RNAi pathway, siRNAs also act in RNAi-related pathways, e.g., as an antiviral mechanism or in shaping the chromatin structure of a genome. Some non limiting examples for target genes, or biological pathways which can be interfered by siRNA are epidermal growth factor receptor variant III gene, which is expressed in 40-50% of gliomas, and the phosphoinositide 3-kinase (PI3K)/Akt pathway, which plays a crucial role in medulloblastoma biology. In other aspects of this embodiment, the polynucleotide is a DNA-RNA hybrid molecule.

In certain embodiments, the bolaamphiphile vesicle complexes comprise one or more bolaamphiphilic compounds and the biologically active compound is a siRNA that is a mixture of two or more siRNA, wherein at least one siRNA is directed to a first target, and at least one siRNA is directed to a second target.

In further embodiments, provided herein are novel siRNA and bolamphiphilic vesicle complex comprising siRNA and one or more bolaamphiphilic compounds.

In further embodiments, provided herein are novel formulations of siRNA with bolaamphiphilic compounds or with bolaamhphilic vesicles.

In another embodiment, provided here are methods of delivering siRNA into animal or human cells.

In an additional embodiment of the disclosure is directed to delivery of siRNA-bolaamphiphile vesicle complexes or siRNA-bolaamphiphilic vesicle complexes into animals or human wherein the bolaamphiphile vesicle complex comprises one or more bolaamphiphilic compounds and siRNA.

In another aspect, provided here are methods of delivering siRNA into animal or human cell comprising the step of administering to the animal or human a pharmaceutical composition comprising of a bolaamphiphile vesicle complex; and wherein the bolaamphiphile vesicle complex comprises one or more bolaamphiphilic compounds and siRNA. In one embodiment, the cell is brain cell, liver cell, gall bladder, or a lung cell. In other embodiments, the cells are are cells of a lymph node, a CD4+ lymphocyte, or a cell of the mononuclear phagocyte system, including, without limitation, a monocyte, macrophage, a resident brain microglial cell and a dendritic cell. In a still further emobidment, the cell is a cancer cell.

In another aspect, provided here are methods of delivering siRNA into animal or human organs comprising the step of administering to the animal or human a pharmaceutical composition comprising of a bolaamphiphile vesicle complex; and wherein the bolaamphiphile vesicle complex comprises one or more bolaamphiphilic compounds and siRNA. In one embodiment, the organ is brain, liver, gall bladder, a lymph node or a lung. In certain aspects of this emobidment, the siRNA is delivered to a tumor.

In a further embodiment the active agent is an RNA-DNA heteroduplex with properties of siRNA molecules. In certain aspects of this embodiment, the bolaamphiphile vesicle complexes comprise one or more bolaamphiphilic compounds and the biologically active compound is a siRNA that is a mixture of two or more siRNA or a mixture comprising at least one siRNA and one RNA-DNA duplex, wherein at least one siRNA or RNA-DNA duplex is directed to a first target, and at least one siRNA or RNA-DNA duplex is directed to a second target.

In certain embodiments, the target is a promoter. In other emodiments, the first and second targets are sequences of separate and distinct genes.

In another specific aspect, provided herein are pharmaceutical compositions, comprising a nano-sized particle comprising one or more bolaamphiphilic compounds and siRNA; and a pharmaceutically acceptable carrier.

In another specific aspect, provided herein are methods for treatment or diagnosis of diseases or disorders selected from cancer such as breast cancer, prostate cancer and brain tumors using the nano-particles, pharmaceutical compositions or formulations of the present invention.

**** In another specific aspect, provided herein are methods for delivering siRNA across the cell membrane. In one embodiment, the cell is brain cell, liver cell, gall bladder cell, or a lung cell. In other specific aspects, the cells are are cells of a lymph node, a CD4+ lymphocyte, or a cell of the mononuclear phagocyte system, including, without limitation, a monocyte, macrophage, a resident brain microglial cell and a dendritic cell.

In another aspect, provided here are methods of delivering one or more biologically active compounds selected from among basic amino acids (e.g., histidine), mRNA molecules, antisense oligonucleotides, peptide targeting ligands, and combinations thereof into animal or human brain comprising the step of administering to the animal or human a pharmaceutical composition comprising of a bolaamphiphile vesicle complex; and wherein the bolaamphiphile vesicle complex comprises one or more bolaamphiphilic compounds and one or more biologically active compounds selected from among basic amino acids (e.g., histidine), mRNA molecules, antisense oligonucleotides, peptide targeting ligands, and combinations thereof.

In another aspect, provided here are methods of delivering one or more biologically active compounds selected from among basic amino acids (e.g., histidine), mRNA molecules, antisense oligonucleotides, peptide targeting ligands, and combinations thereof into animal or human liver comprising the step of administering to the animal or human a pharmaceutical composition comprising of a bolaamphiphile vesicle complex; and wherein the bolaamphiphile vesicle complex comprises one or more bolaamphiphilic compounds and one or more biologically active compounds selected from among basic amino acids (e.g., histidine), mRNA molecules, antisense oligonucleotides, peptide targeting ligands, and combinations thereof.

In another aspect, provided here are methods of delivering one or more biologically active compounds selected from among basic amino acids (e.g., histidine), mRNA molecules, antisense oligonucleotides, peptide targeting ligands, and combinations thereof into animal or human lungs comprising the step of administering to the animal or human a pharmaceutical composition comprising of a bolaamphiphile vesicle complex; and wherein the bolaamphiphile vesicle complex comprises one or more bolaamphiphilic compounds and one or more biologically active compounds selected from among basic amino acids (e.g., histidine), mRNA molecules, antisense oligonucleotides, peptide targeting ligands, and combinations thereof.

In another aspect, provided here are methods of delivering one or more biologically active compounds selected from among basic amino acids (e.g., histidine), mRNA molecules, antisense oligonucleotides, peptide targeting ligands, and combinations thereof into animal or human gall bladder comprising the step of administering to the animal or human a pharmaceutical composition comprising of a bolaamphiphile vesicle complex; and wherein the bolaamphiphile vesicle complex comprises one or more bolaamphiphilic compounds and one or more biologically active compounds selected from among basic amino acids (e.g., histidine), mRNA molecules, antisense oligonucleotides, peptide targeting ligands, and combinations thereof.

In another specific aspect, provided herein are nano-particles, comprising one or more bolaamphiphilic compounds and one or more biologically active compounds selected from among basic amino acids (e.g., histidine), mRNA molecules, antisense oligonucleotides, peptide targeting ligands, and combinations thereof. In one embodiment, the bolaamphiphilic compounds and siRNA are encapsulated within the nano-particle.

In particular embodiments, one or more biologically active compounds selected from among basic amino acids (e.g., histidine), mRNA molecules, antisense oligonucleotides, peptide targeting ligands, and combinations thereof are delivered by the nanoparticles of the invention. In a more particular embodiment, the polynucleotide is an antisense oligonucleotides, of 20-25 nucleotides. Some non limiting examples for target genes, or biological pathways which can be interfered by antisesense oligonucleotides are epidermal growth factor receptor variant III gene, which is expressed in 40-50% of gliomas, and the phosphoinositide 3-kinase (PI3K)/Akt pathway, which plays a crucial role in medulloblastoma biology.

In further embodiments, provided herein are novel formulations of one or more biologically active compounds selected from among basic amino acids (e.g., histidine), mRNA molecules, antisense oligonucleotides, peptide targeting ligands, and combinations thereof, with bolaamphiphilic compounds or with bolaamhphilic vesicles.

In another embodiment, provided here are methods of delivering one or more biologically active compounds selected from among basic amino acids (e.g., histidine), mRNA molecules, antisense oligonucleotides, peptide targeting ligands, and combinations thereof into animal or human cells.

In an additional embodiment of the disclosure is directed to delivery of biologically-active-material-bolaamphiphile vesicle complexes or biologically-active material-bolaamphiphilic vesicle complexes into animals or human wherein the bolaamphiphile vesicle complex comprises one or more bolaamphiphilic compounds and one or more biologically active compounds selected from among basic amino acids (e.g., histidine), mRNA molecules, antisense oligonucleotides, peptide targeting ligands, and combinations thereof.

In another aspect, provided here are methods of delivering siRNA into animal or human cell comprising the step of administering to the animal or human a pharmaceutical composition comprising of a bolaamphiphile vesicle complex; and wherein the bolaamphiphile vesicle complex comprises one or more bolaamphiphilic compounds and one or more biologically active compounds selected from among basic amino acids (e.g., histidine), mRNA molecules, antisense oligonucleotides, peptide targeting ligands, and combinations thereof. In one embodiment, the cell is brain cell, liver cell, gall bladder, or a lung cell. In other embodiments, the cells are are cells of a lymph node, a CD4+ lymphocyte, or a cell of the mononuclear phagocyte system, including, without limitation, a monocyte, macrophage, a resident brain microglial cell and a dendritic cell. In a still further emobidment, the cell is a cancer cell.

In another aspect, provided here are methods of delivering one or more biologically active compounds selected from among basic amino acids (e.g., histidine), mRNA molecules, antisense oligonucleotides, peptide targeting ligands, and combinations thereof into animal or human organs comprising the step of administering to the animal or human a pharmaceutical composition comprising of a bolaamphiphile vesicle complex; and wherein the bolaamphiphile vesicle complex comprises one or more bolaamphiphilic compounds and one or more biologically active compounds selected from among basic amino acids (e.g., histidine), mRNA molecules, antisense oligonucleotides, peptide targeting ligands, and combinations thereof. In one embodiment, the organ is brain, liver, gall bladder, a lymph node or a lung. In certain aspects of this emobidment, the one or more biologically active compounds selected from among basic amino acids (e.g., histidine), mRNA molecules, antisense oligonucleotides, peptide targeting ligands, and combinations thereof is delivered to a tumor.

In another specific aspect, provided herein are pharmaceutical compositions, comprising a nano-sized particle comprising one or more bolaamphiphilic compounds and one or more biologically active compounds selected from among basic amino acids (e.g., histidine), mRNA molecules, antisense oligonucleotides, peptide targeting ligands, and combinations thereof, and a pharmaceutically acceptable carrier. In other, specific aspects of these embodiment, the peptide targeting ligand is based upon or derived from the tetanus toxin, providing a ligand for neurospecific binding.

In another specific aspect, provided herein are methods for treatment or diagnosis of diseases or disorders selected from cancer such as breast cancer, prostate cancer and brain tumors using the nano-particles, pharmaceutical compositions or formulations of the present invention.

The present disclosure is further directed to methods of delivering bolaamphiphile vesicle complexes disclosed comprise one or more bolaamphiphilic compounds and one or more biologically active compounds selected from among basic amino acids (e.g., histidine), mRNA molecules, antisense oligonucleotides, and peptide targeting ligands

In another aspect, provided herein are methods for delivering basic amino acids (e.g., histidine), mRNA molecules, antisense oligonucleotides, and peptide targeting ligands into animal or human organs comprising the step of administering to the animal or human a pharmaceutical composition comprising a bolaamphiphile vesicle complex; and wherein the bolaamphiphile vesicle complex comprises one or more bolaamphiphilic compounds and a biologically active compound selected from among basic amino acids (e.g., histidine), mRNA molecules, antisense oligonucleotides, peptide targeting ligands and combinations thereof. In one embodiment, the organ is brain, liver, gall bladder, a lymph node or a lung. In certain aspects of this emobidment, the biologically active compound, selected from among basic amino acids (e.g., histidine), mRNA molecules, antisense oligonucleotides, peptide targeting ligands and combinations thereof, is delivered to a tumor. In other aspects of this embodiment, the compositions are delivered to other organs, tissue, and cells as described hererin.

In certain aspects of the present disclosure, the siRNA and/or antisense oliogonucleotide (e.g., but not limited to antisense c-fos, c-myc, K-ras), may be directed against genes that control the cell cycle or signaling pathways. In other aspects, the nano-particle may also carry one or more antineoplastic drugs, including but not limited to, adriamycin, angiostatin, azathioprine, bleomycin, busulfane, camptothecin, carboplatin, carmustine, chlorambucile, chlormethamine, chloroquinoxaline sulfonamide, cisplatin, cyclophosphamide, cycloplatam, cytarabine, dacarbazine, dactinomycin, daunorubicin, didox, doxorubicin, endostatin, enloplatin, estramustine, etoposide, extramustinephosphat, flucytosine, fluorodeoxyuridine, fluorouracil, gallium nitrate, hydroxyurea, idoxuridine, interferons, interleukins, leuprolide, lobaplatin, lomustine, mannomustine, mechlorethamine, mechlorethaminoxide, melphalan, mercaptopurine, methotrexate, mithramycin, mitobronitole, mitomycin, mycophenolic acid, nocodazole, oncostatin, oxaliplatin, paclitaxel, pentamustine, platinum-triamine complex, plicamycin, prednisolone, prednisone, procarbazine, protein kinase C inhibitors, puromycine, Semustine, signal transduction inhibitors, spiroplatin, streptozotocine, stromelysin inhibitors, taxol, tegafur, telomerase inhibitors, teniposide, thalidomide, thiamiprine, thioguanine, thiotepa, tiamiprine, tretamine, triaziquone, trifosfamide, tyrosine kinase inhibitors, uramustine, vidarabine, vinblastine, vinca alcaloids, vincristine, Vindesine, vorozole, zeniplatin, zeniplatin, zinostatin, and combinations thereof.

The antisense oligonucleotides may have some or all of the nucleotide linkages substituted with stable, non-phosphodiester linkages, including, for example, phosphorothioate, phosphorodithioate, phosphoroselenate, methylphosphonate, or O-alkyl phosphotriester linkages.

The present disclosure further provides bolaamphiphiles with histidine head groups. In one aspect of this embodiment, the alkyl chain is connected though the amine group of the imidazole of the histidine, providing cationic bolaamphiphiles with enhanced penetration through biological barriers such as the brain blood barrier (BBB). The alpha amino acid groups may undergo decarboxylation at given sites which lead to reorganization of the bolaamphiphile aggregate structures and release of encapsulated agents at the site of hydrolysis. In another aspect of this embodiment, the histidine groups may be attached to the alkyl chain through the carboxyl groups. Aggregate structures may also have enhanced transport through biological barriers because of the imidazole and since the conjugate acid (protonated form) of the imidazole side chain in histidine has a pKa of approximately 6.0 physiologically relevant pH values, relatively small shifts in pH will change its average charge and below a pH of 6, the imidazole ring is mostly protonated which would selectively disrupt the vesicles at this site and release the active agent.

In another embodiment, the bola aggregates disclosed herein are useful for the delivery of messenger RNA (mRNA) to specific sites and in one important embodiment to sites in the CNS that require penetration through neural blood barriers such as the blood brain barrier. mRNA is a large family of RNA molecules that convey genetic information from DNA to the ribosome, where they specify the amino acid sequence of the protein products of gene expression. Following transcription of primary transcript mRNA Mature mRNA is translated into a polymer of amino acids: a protein, as summarized in the central dogma of molecular biology. By delivery of mRNA by the invented targeted delivery systems disease states such as but not limited to brain tumors and cancers, bacterial and viral and other microbial infections, and metabolic disorders may be treated (e.g., diabeties).

The nano vesicles and bola complexes of the disclosure may be used to deliver mRNA to hepatocyte cells of the main tissue of the liver in order to control the following process for the prevention and treatment of diseases in which the following said processes are involved: protein synthesis, protein storage, carbohydrate transformation, synthesis of cholesterol, bile salts, and phospholipids, and detoxification, modification, and excretion of exogenous and endogenous substances.

In a further embodiment, the present disclosure provides targeted delivery of Antisense oligonucleotides to specific sites and in one important embodiment to sites with neural blood barriers such as the CNS or brain which requires intact penetration across the BBB. Antisense oligonucleotides have many important application embodiments in the prevention and treatment of different diseases and disorders. Antisense oligonucleotides are single strands of DNA or RNA that are complementary to a chosen sequence. In the case of antisense RNA they prevent protein translation of certain messenger RNA strands by binding to them. Antisense DNA can be used to target a specific, complementary (coding or non-coding) RNA. If binding takes place this DNA/RNA hybrid can be degraded by the enzyme RNase H.

Antisense oligonucleotides can be used as therapeutic agents that interfere with and block disease processes by altering the synthesis of a particular protein, by the binding of the antisense oligonucleotide to the mRNA from which that protein is normally synthesized. Binding of the two may physically block the ability of ribosomes to move along the messenger RNA preventing synthesis of the protein; hasten the rate at which the mRNA is degraded within the cytosol; and prevent splicing errors that would otherwise produce a defective protein. However, in order to be useful in human therapy, antisense oligonucleotides must be able to enter the target cells; avoid digestion by nucleases; and not cause dangerous side-effects. In order to achieve these goals, antisense oligonucleotides are encapsulated or complexed within the bolaamphiphile complexes and/or nano-vesicles of the disclosure, which can then resist digestion by nucleases; and be targeted to a given site using the ligand for the type of receptors found on desired target cells on the surface of the nano vesicles; antibodies decorating the surface of the nano particles directed against molecules on the surface of the desired target cells.

In certain embodiments, examples of diseased states that can be treated by the presently-disclosed delivery of mRNA in the nano-vesicles and complexes of the disclosure include, for example, Hepatitis C virus (HCV) (successful infection of the liver by HCV requires that the liver produce a particular microRNA (miRNA-122). Injections of HCV-infected humans with an ODN (“miravirsen”) complementary to miRNA-122 suppresses the virus); HIV-1, the most frequent cause of AIDS in the United States; Ebola virus (the cause of the often-fatal Ebola hemorrhagic fever); human cytomegalovirus (HCMV) (which frequently causes serious complications in AIDS patients); asthma (inhalation of an antisense oligonucleotide reduces the synthesis of cell receptors involved in asthma in at least one model system); certain cancers, (e.g., chronic myelogenous leukemia (CML); certain types of inflammation caused by cell-mediated immune reactions; Duchenne muscular dystrophy (DMD); familial hypercholesterolemia (targets the mRNA for apolipoprotein B-100; e.g., On 31 Jan. 2013, the antisense ODN mipomersen (Kynamro®) received regulatory approval for use in humans with familial hypercholesterolemia).

The present disclosure also provides novel nano vesicles with surface decorated with peptides with the binding characteristics of tetanus toxin showed strong binding to PC 12, primary motor neurons, and dorsal root ganglion (DRG) cells. The enhanced neuronal binding affinity and specificity of peptide targeting ligands to tetanus, has application for targeting neurotherapeutic proteins and viral vectors in the treatment of motor neuron disease, neuropathy, and pain. In one embodiment comprises the use of peptide targeting ligand to tetanus on motor neurons as application in delivery of SiRNA or NTF such as GDNF for the treatment of ALS. In one non limiting embodiment, the novel peptide is that described in Neurobiol Dis. 2005 August; 19(3): 407-18 by Liu et al., has the binding characteristics of tetanus toxin has application in therapeutic protein and vector motor and sensory neuron targeting.

In another embodiment, the enhanced neuronal binding affinity and specificity of Tet1, a novel 12 amino acid peptide, is used for targeting neurotherapeutic proteins and viral vectors in the treatment of motor neuron disease, neuropathy, and pain. The advantage of using the delivery systems based on bolaamphilies nano-vesicles and/or complexes disclose herein comprising the described targeting ligands is improved stability, penetration through biological barriers and selective release at the target site. That is, the tetanus targeting ligand is but one example of the other peptides with the binding characteristics of tetanus toxin.

Accordingly, in one embodiment, the present disclosure provides bolaamphiphiles with histidine head groups and bola nano-vesicles and complexes for delivery of active agents that include/comprise the said bolaamphiphile with the said histidine head groups.

In another embodiment, the present disclosure provides bola nano-vesicles and complexes with mRNA for delivery to sites including but not limited to the brain, for treatment of diseases and disorders as described herein.

In a further embodiment, the present disclosure also provides a method for delivery to the brain after systemic administration of the bolaamphiphile aggregates and nano vesicles comprising a therapeutic agent.

In another embodiment, the present disclosure provides a method for delivery of bolaamphiphile aggregates and nano vesicles comprising a therapeutic agent of the disclosure to hepatocyte cells of the liver and the management of the diseases or disorder described above.

In a further embodiment, the present disclosure provides bolaamphiphile nano-vesicles and complexes with Antisense oligonucleotides for delivery to sites such as but not limited to the brain for treatment of diseases and disorders as described above.

In a still further embodiment, the present disclosure also provides a method for the delivery of therapeutic bolaamphiphile nano-vesicles and complexes of the disclosure to the brain after systemic administration.

In a further embodiment, the present disclosure also provides bolaamphiphile nano-vesicles and complexes having all or part of a surface decorated with peptides having the binding characteristics of tetanus toxin. In one aspect of this embodiment, these aggregate structures are administered for treatment of motor neuron disease, neuropathy, and pain, e.g., in one non limiting aspect, treatment of amyotrophic lateral sclerosis (ALS).

The Derivatives and Precursors disclosed can be prepared as illustrated in the Schemes provided herein. The syntheses can involve initial construction of, for example, vemonia oil or direct functionalization of natural derivatives by organic synthesis manipulations such as, but not limiting to, epoxide ring opening. In those processes involving oxiranyl ring opening, the epoxy group is opened by the addition of reagents such as carboxylic acids or organic or inorganic nucleophiles. Such ring opening results in a mixture of two products in which the new group is introduced at either of the two carbon atoms of the epoxide moiety. This provides beta substituted alcohols in which the substitution position most remote from the CO group of the main aliphatic chain of the vemonia oil derivative is arbitrarily assigned as position 1, while the neighboring substituted carbon position is designated position 2. For simplicity purposes only, the Derivatives and Precursors shown herein may indicate structures with the hydroxy group always at position 2 but the Derivatives and Precursors wherein the hydroxy is at position 1 are also encompassed by the invention. Thus, a radical of the formula —CH(OH)—CH(R) refers to the substitution of —OH at either the carbon closer to the CO group, designated position 2 or to the carbon at position 1. Moreover, with respect to the preparation of symmetrical bolaamphiphiles made via introducing the head groups through an epoxy moiety (e.g., as in vernolic acid) or a double bond (—C═C—) as in mono unsaturated fatty acids (e.g., oleic acid) a mixture of three different derivatives will be produced. In certain embodiments, vesicles are prepared using the mixture of unfractionated positional isomers. In one aspect of this embodiment, where one or more bolas are prepared from vernolic acid, and in which a hydroxy group as well as the head group introduced through an epoxy or a fatty acid with the head group introduced through a double bond (—C═C—), the bola used in vesicle preparation can actually be a mixture of three different positional isomers.

In other embodiments, the three different derivatives are isolated. Accordingly, the vesicles disclosed herein can be made from a mixture of the three isomers of each derivative or, in other embodiments, the individual isomers can be isolated and used for preparation of vesicles.

Symmetrical bolaamphiphiles can form relatively stable self aggregate vesicle structures by the use of additives such as cholesterol and cholesterol derivatives (e.g., cholesterol hemisuccinate, cholesterol oleyl ether, anionic and cationic derivatives of cholesterol and the like), or other additives including single headed amphiphiles with one, two or multiple aliphatic chains such as phospholipids, zwitterionic, acidic, or cationic lipids. Examples of zwitterionic lipids are phosphatidylcholines, phosphatidylethanol amines and sphingomyelins. Examples of acidic amphiphilic lipids are phosphatidylglycerols, phosphatidylserines, phosphatidylinositols, and phosphatidic acids. Examples of cationic amphipathic lipids are diacyl trimethylammonium propanes, diacyl dimethylammonium propanes, and stearylamines cationic amphiphiles such as spermine cholesterol carbamates, and the like, in optimum concentrations which fill in the larger spaces on the outer surfaces, and/or add additional hydrophilicity to the particles. Such additives may be added to the reaction mixture during formation of nanoparticles to enhance stability of the nanoparticles by filling in the void volumes of in the upper surface of the vesicle membrane.

Stability of nano vesicles according to the present disclosure can be demonstrated by dynamic light scattering (DLS) and transmission electron microscopy (TEM). For example, suspensions of the vesicles can be left to stand for 1, 5, 10, and 30 days to assess the stability of the nanoparticle solution/suspension and then analyzed by DLS and TEM.

The vesicles disclosed herein may encapsulate within their core the active agent, which in particular embodiments is selected from peptides, proteins, nucleotides and or non-polymeric agents. In certain embodiments, the active agent is also associated via one or more non-covalent interactions to the vesicular membrane on the outer surface and/or the inner surface, optionally as pendant decorating the outer or inner surface, and may further be incorporated into the membrane surrounding the core. In certain aspects, biologically active peptides, proteins, nucleotides or non-polymeric agents that have a net electric charge, may associate ionically with oppositely charged headgroups on the vesicle surface and/or form salt complexes therewith.

In particular aspects of these embodiments, additives which may be bolaamphiphiles or single headed amphiphiles, comprise one or more branching alkyl chains bearing polar or ionic pendants, wherein the aliphatic portions act as anchors into the vesicle's membrane and the pendants (e.g., chitosan derivatives or poly amines or certain peptides) decorate the surface of the vesicle to enhance penetration through various biological barriers such as the intestinal tract and the BBB, and in some instances are also selectively hydrolyzed at a given site or within a given organ. The concentration of these additives is readily adjusted according to experimental determination.

In certain embodiments, the oral formulations of the present disclosure comprise agents that enhance penetration through the membranes of the GI tract and enable passage of intact nanoparticles containing the drug. These agents may be any of the additives mentioned above and, in particular aspects of these embodiment, include chitosan and derivatives thereof, serving as vehicle surface ligands, as decorations or pendants on the vesicles, or the agents may be excipients added to the formulation.

In other embodiments, the nanoparticles and vesicles disclosed herein may comprise the fluorescent marker carboxyfluorescein (CF) encapsulated therein while in particular aspects, the nanoparticle and vesicles of the present disclosure may be decorated with one or more of PEG, e.g. PEG2000-vemonia derivatives as pendants. For example, two kinds of PEG-vemonia derivatives can be used: PEG-ether derivatives, wherein PEG is bound via an ether bond to the oxygen of the opened epoxy ring of, e.g., vernolic acid and PEG-ester derivatives, wherein PEG is bound via an ester bond to the carboxylic group of, e.g., vernolic acid.

In other embodiments, vesicles, made from synthetic amphiphiles, as well as liposomes, made from synthetic or natural phospholipids, substantially (or totally) isolate the therapeutic agent from the environment allowing each vesicle or liposome to deliver many molecules of the therapeutic agent. Moreover, the surface properties of the vesicle or liposome can be modified for biological stability, enhanced penetration through biological barriers and targeting, independent of the physico-chemical properties of the encapsulated drug.

In still other embodiments, the headgroup is selected from: (i) choline or thiocholine, O-alkyl, N-alkyl or ester derivatives thereof; (ii) non-aromatic amino acids with functional side chains such as glutamic acid, aspartic acid, lysine or cysteine, or an aromatic amino acid such as tyrosine, tryptophan, phenylalanine and derivatives thereof such as levodopa (3,4-dihydroxy-phenylalanine) and p-aminophenylalanine; (iii) a peptide or a peptide derivative that is specifically cleaved by an enzyme at a diseased site selected from enkephalin, N-acetyl-ala-ala, a peptide that constitutes a domain recognized by beta and gamma secretases, and a peptide that is recognized by stromelysins; (iv) saccharides such as glucose, mannose and ascorbic acid; and (v) other compounds such as nicotine, cytosine, lobeline, polyethylene glycol, a cannabinoid, or folic acid.

In further embodiments, nano-sized particle and vesicles disclosed herein further comprise at least one additive for one or more of targeting purposes, enhancing permeability and increasing the stability the vesicle or particle. Such additives, in particular aspects, may selected from from: (i) a single headed amphiphilic derivative comprising one, two or multiple aliphatic chains, preferably two aliphatic chains linked to a midsection/spacer region such as —NH—(CH₂)₂—N—(CH₂)₂—N—, or —O—(CH₂)₂—N—(CH₂)₂—O—, and a sole headgroup, which may be a selectively cleavable headgroup or one containing a polar or ionic selectively cleavable group or moiety, attached to the N atom in the middle of said midsection. In other asepcts, the additive can be selected from among cholesterol and cholesterol derivatives such as cholesteryl hemmisuccinate; phospholipids, zwitterionic, acidic, or cationic lipids; chitosan and chitosan derivatives, such as vernolic acid-chitosan conjugate, quaternized chitosan, chitosan-polyethylene glycol (PEG) conjugates, chitosan-polypropylene glycol (PPG) conjugates, chitosan N-conjugated with different amino acids, carboxy alkylated chitosan, sulfonyl chitosan, carbohydrate-branched N-(carboxymethylidene) chitosan and N-(carboxymethyl) chitosan; polyamines such as protamine, polylysine or polyarginine; ligands of specific receptors at a target site of a biological environment such as nicotine, cytisine, lobeline, 1-glutamic acid MK801, morphine, enkephalins, benzodiazepines such as diazepam (valium) and librium, dopamine agonists, dopamine antagonists tricyclic antidepressants, muscarinic agonists, muscarinic antagonists, cannabinoids and arachidonyl ethanol amide; polycationic polymers such as polyethylene amine; peptides that enhance transport through the BBB such as OX 26, transferrins, polybrene, histone, cationic dendrimer, synthetic peptides and polymyxin B nonapeptide (PMBN); monosaccharides such as glucose, mannose, ascorbic acid and derivatives thereof; modified proteins or antibodies that undergo absorptive-mediated or receptor-mediated transcytosis through the blood-brain barrier, such as bradykinin B2 agonist RMP-7 or monoclonal antibody to the transferrin receptor; mucoadhesive polymers such as glycerides and steroidal detergents; and Ca²⁺ chelators. The aforementioned head groups on the additives designed for one or more of targeting purposes and enhancing permeability may also be a head group, preferably on an asymmetric bolaamphiphile wherein the other head group is another moiety, or the head group on both sides of a symmetrical bolaamphiphile.

In other embodiments, nano-sized particle and vesicles discloser herein may comprises at least one biologically active agent is selected from: (i) a natural or synthetic peptide or protein such as analgesics peptides from the enkephalin class, insulin, insulin analogs, oxytocin, calcitonin, tyrotropin releasing hormone, follicle stimulating hormone, luteinizing hormone, vasopressin and vasopressin analogs, catalase, interleukin-II, interferon, colony stimulating factor, tumor necrosis factor (TNF), melanocyte-stimulating hormone, superoxide dismutase, glial cell derived neurotrophic factor (GDNF) or the Gly-Leu-Phe (GLF) families; (ii) nucleosides and polynucleotides selected from DNA or RNA molecules such as small interfering RNA (siRNA) or a DNA plasmid; (iii) antiviral and antibacterial; (iv) antineoplastic and chemotherapy agents such as cyclosporin, doxorubicin, epirubicin, bleomycin, cisplatin, carboplatin, vinca alkaloids, e.g. vincristine, Podophyllotoxin, taxanes, e.g. Taxol and Docetaxel, and topoisomerase inhibitors, e.g. irinotecan, topotecan.

Additional embodiments within the scope provided herein are set forth in non-limiting fashion elsewhere herein and in the examples. It should be understood that these examples are for illustrative purposes only and are not to be construed as limiting in any manner.

### PHARMACEUTICAL COMPOSITIONS

In another aspect, the invention provides a pharmaceutical composition comprising a pharmaceutically acceptable carrier and a pharmaceutically effective amount of a compound of Formula I or a complex thereof.

When employed as pharmaceuticals, the compounds provided herein are typically administered in the form of a pharmaceutical composition. Such compositions can be prepared in a manner well known in the pharmaceutical art and comprise at least one active compound.

In certain embodiments, with respect to the pharmaceutical composition, the carrier is a parenteral carrier, oral or topical carrier.

The present invention also relates to a compound or pharmaceutical composition of compound according to Formula I; or a pharmaceutically acceptable salt or solvate thereof for use as a pharmaceutical or a medicament.

Generally, the compounds provided herein are administered in a therapeutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions provided herein can be administered by a variety of routes including oral, rectal, transdermal, subcutaneous, intravenous, intramuscular, and intranasal. Depending on the intended route of delivery, the compounds provided herein are preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pa., which is incorporated herein by reference.

The above-described components for orally administrable, injectable, or topically administrable compositions are merely representative. Other materials as well as processing techniques and the like are set forth in Part 8 of Remington's The Science and Practice of Pharmacy, 21st edition, 2005, Publisher: Lippincott Williams & Wilkins, which is incorporated herein by reference.

The compounds of this invention can also be administered in sustained release forms or from sustained release drug delivery systems. A description of representative sustained release materials can be found in Remington's Pharmaceutical Sciences.

The present invention also relates to the pharmaceutically acceptable salts of compounds of Formula I.

The following formulation examples illustrate representative pharmaceutical compositions that may be prepared in accordance with this invention. The present invention, however, is not limited to the following pharmaceutical compositions.

### Formulation 1—Injection

A compound of the invention may be dissolved or suspended in a buffered sterile saline injectable aqueous medium to a concentration of approximately 5 mg/mL.

### Methods of Treatment

Bolaamphiphilic vesicles (bolavesicles) may have certain advantages over conventional liposomes as potential vehicles for drug delivery. Bolavesicles have thinner membranes than comparable liposomal bilayer, and therefore possess bigger inner volume and hence higher encapsulation capacity than liposomes of the same diameter. Moreover, bolavesicles are more physically-stable than conventional liposomes, but can be destabilized in a triggered fashion (e.g., by hydrolysis of the headgroups using a specific enzymatic reaction) thus allowing controlled release of the encapsulated material at the site of action (i.e., drug targeting).

Specific small interfering RNAs (siRNAs) designed to silence different oncogenic pathways can be used for cancer therapy. However, in the blood stream, non-modified naked non-modified siRNAs are unstable, thus having a short half-life in the blood stream and encounter difficulties in crossing biological membranes due to their negative charge. Therefore, siRNAs may not be used efficiently to silence genes. These obstacles can be overcome by using siRNAs complexed with bolaamphiphiles, consisting of two positively charged head groups that flank a hydrophobic chain. Bolaamphiphiles have relatively low toxicities, long persistence in the blood stream, and most importantly, can form poly-cationic micelles in aqueous conditions thus, becoming amenable to association with negatively charged siRNAs.

Experiments confirmed the formation of stable complexes the bolaamphiphiles of the present invention those can protect nucleic acids from their degradation and thus effectively deliver siRNAs into the cells causing the silencing of target genes.

### General Synthetic Procedures

The compounds provided herein can be purchased or prepared from readily available starting materials using the following general methods and procedures. See, e.g., Synthetic Schemes below. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include (but are not limited to) recrystallization, column chromatography or HPLC. The following schemes are presented with details as to the preparation of representative substituted biarylamides that have been listed herein. The compounds provided herein may be prepared from known or commercially available starting materials and reagents by one skilled in the art of organic synthesis.

The enantiomerically pure compounds provided herein may be prepared according to any techniques known to those of skill in the art. For instance, they may be prepared by chiral or asymmetric synthesis from a suitable optically pure precursor or obtained from a racemate by any conventional technique, for example, by chromatographic resolution using a chiral column, TLC or by the preparation of diastereoisomers, separation thereof and regeneration of the desired enantiomer. See, e.g., “Enantiomers, Racemates and Resolutions,” by J. Jacques, A. Collet, and S. H. Wilen, (Wiley-Interscience, New York, 1981); S. H. Wilen, A. Collet, and J. Jacques, Tetrahedron, 2725 (1977); E. L. Eliel Stereochemistry of Carbon Compounds (McGraw-Hill, N Y, 1962); and S. H. Wilen Tables of Resolving Agents and Optical Resolutions 268 (E. L. Eliel ed., Univ. of Notre Dame Press, Notre Dame, Ind., 1972, Stereochemistry of Organic Compounds, Ernest L. Eliel, Samuel H. Wilen and Lewis N. Manda (1994 John Wiley & Sons, Inc.), and Stereoselective Synthesis A Practical Approach, Mihály Nógrádi (1995 VCH Publishers, Inc., NY, NY).

In certain embodiments, an enantiomerically pure compound of formula (1) may be obtained by reaction of the racemate with a suitable optically active acid or base. Suitable acids or bases include those described in Bighley et al., 1995, Salt Forms of Drugs and Adsorption, in Encyclopedia of Pharmaceutical Technology, vol. 13, Swarbrick & Boylan, eds., Marcel Dekker, New York; ten Hoeve & H. Wynberg, 1985, Journal of Organic Chemistry 50:4508-4514; Dale & Mosher, 1973, J Am. Chem. Soc. 95:512; and CRC Handbook of Optical Resolution via Diastereomeric Salt Formation, the contents of which are hereby incorporated by reference in their entireties.

Enantiomerically pure compounds can also be recovered either from the crystallized diastereomer or from the mother liquor, depending on the solubility properties of the particular acid resolving agent employed and the particular acid enantiomer used. The identity and optical purity of the particular compound so recovered can be determined by polarimetry or other analytical methods known in the art. The diasteroisomers can then be separated, for example, by chromatography or fractional crystallization, and the desired enantiomer regenerated by treatment with an appropriate base or acid. The other enantiomer may be obtained from the racemate in a similar manner or worked up from the liquors of the first separation.

In certain embodiments, enantiomerically pure compound can be separated from racemic compound by chiral chromatography. Various chiral columns and eluents for use in the separation of the enantiomers are available and suitable conditions for the separation can be empirically determined by methods known to one of skill in the art. Exemplary chiral columns available for use in the separation of the enantiomers provided herein include, but are not limited to CHIRALCEL® OB, CHIRALCEL® OB-H, CHIRALCEL® OD, CHIRALCEL® OD-H, CHIRALCEL® OF, CHIRALCEL® OG, CHIRALCEL® OJ and CHIRALCEL® OK.

### Abbreviations

BBB, blood brain barrier

BCECs, brain capillary endothelial cells

CF, carboxyfluorescein

CHEMS, cholesteryl hemisuccinate

CHOL, cholesterol

Cryo-TEM, Cryo-transmission electron microscope

DAPI, 4′,6-diamidino-2-phenylindole

DDS, drug delivery system

DLS, dynamic light scattering

DMPC, 1,2-dimyristoyl-sn-glycero-3-phosphocholine

DMPE, 1,2-dimyristoyl-sn-glycero-3-phosphoethanolamine

DMPG, 1,2-dimyristoyl-sn-glycero-3-phospho-(1′-rac-glycerol)

EPR, electron paramagnetic resonance

FACS, fluorescence-activated cell sorting

FCR, fluorescence colorimetric response

GUVs, giant unilamellar vesicles

HPLC, high performance liquid chromatography

IR, infrared

MNPs, Magnetic Nanoparticles

MRI, magnetic resonance imaging

NMR, nuclear magnetic resonance

NPs, nanoparticles

PBS, phosphate buffered saline

PC, phosphatidylcholine

PDA, polydiacetylene.

TMA-DPH, 1-(4 trimethylammoniumphenyl)-6-phenyl-1,3,5-hexatriene

### Example 1

### Bolaamphiphile Synthesis

The boloamphiphles or bolaamphiphilic compounds of the invention can be synthesized following the procedures described previously (see below).

Briefly, the carboxylic group of methyl vemolate or vemolic acid was interacted with aliphatic diols to obtain bisvemolesters. Then the epoxy group of the vemolate moiety, located on C12 and C13 of the aliphatic chain of vemolic acid, was used to introduce two ACh headgroups on the two vicinal carbons obtained after the opening of the oxirane ring. For GLH-20 (Table 1), the ACh head group was attached to the vemolate skeleton through the nitrogen atom of the choline moiety. The bolaamphiphile was prepared in a two-stage synthesis: First, opening of the epoxy ring with a haloacetic acid and, second, quaternization with the N,N-dimethylamino ethyl acetate. For GLH-19 (Table 1) that contains an ACh head group attached to the vemolate skeleton through the acetyl group, the bolaamphiphile was prepared in a three-stage synthesis, including opening of the epoxy ring with glutaric acid, then esterification of the free carboxylic group with N,N-dimethyl amino ethanol and the final product was obtained by quaternization of the head group, using methyl iodide followed by exchange of the iodide ion by chloride using an ion exchange resin.

Each bolaamphiphile was characterized by mass spectrometry, NMR and IR spectroscopy. The purity of the two bolaamphiphiles was >97% as determined by HPLC.

Materials: Diphenyl ether, 1,2-hexadecanediol, oleic acid, oleylamine, and carboxyfluorescein (CF) were purchased from Sigma Aldrich (Rehovot, Israel). Chloroform and ethanol were purchased from Bio-Lab Ltd. Jerusalem, Israel. 1,2-dimyristoyl-sn-glycero-3-phospho-(1′-rac-glycerol) (DMPG), 1,2-dimyristoyl-sn-glycero-3-phosphoethanolamine (DMPE), 1,2-dimyristoyl-sn-glycero-3-phosphocholine (DMPC), cholesterol (CHOL), cholesteryl hemisuccinate (CHEMS) were purchased from Avanti Lipids (Alabaster, Ala., USA), The diacetylenic monomer 10,12-tricosadiynoic acid was purchased from Alfa Aesar (Karlsruhe, Germany), and purified by dissolving the powder in chloroform, filtering the resulting solution through a 0.45 μm nylon filter (Whatman Inc., Clifton, N.J., USA), and evaporation of the solvent. 1-(4 trimethylammoniumphenyl)-6-phenyl-1,3,5-hexatriene (TMA-DPH) was purchased from Molecular Probes Inc. (Eugene, Oreg., USA).

### Synthesis of Representative Bolaamphiphilic Compounds

The synthesis of bolaamphiphilic compounds of this invention can be carried out in accordance with the methods described previously (Chemistry and Physics of Lipids 2008, 153, 85-97; Journal of Liposome Research 2010, 20, 147-59; WO2002/055011; WO2003/047499; or WO2010/128504) and using the appropriate reagents, starting materials, and purification methods known to those skilled in the art. Several representative bolaamphiphilic compounds of the invention, which are prepared in according the methods described herein or can be prepared following the methods described in the literature or following the methods known to those skilled in the art, are given in Table 1.

TABLE 1
Representative Bolaamphiphiles

| # | Structure |
|---|---|
| GLH-3 | 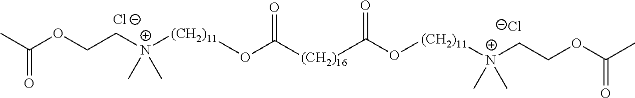 |
| GLH-4 | 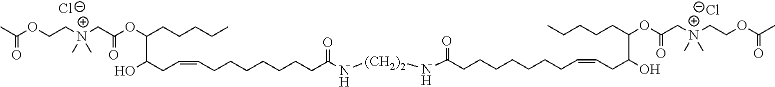 |
| GLH-5 | 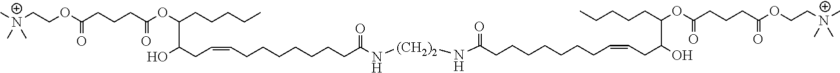 |
| GLH-6^{a} | 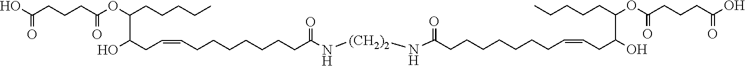 |
| GLH-7 | 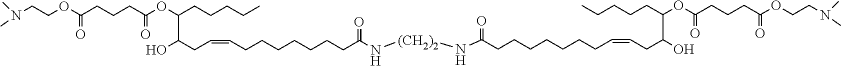 |
| GLH-8* | 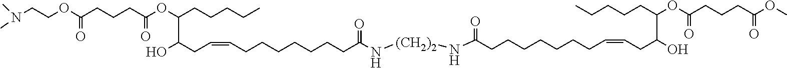 |
| GLH-9 | 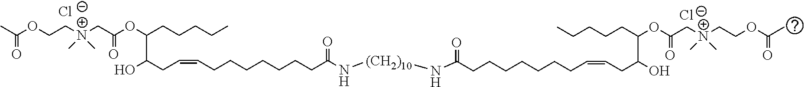 |
| GLH-10 | 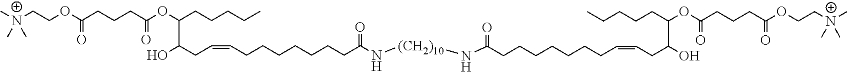 |
| GLH-11 | 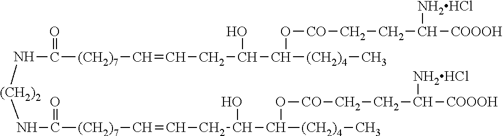 |
| GLH-12^{a} | 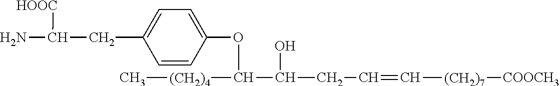 |
| GLH-13^{a} GLH-13^{a} | 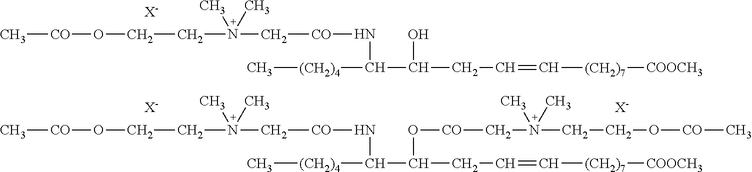 |
| GLH-14 | 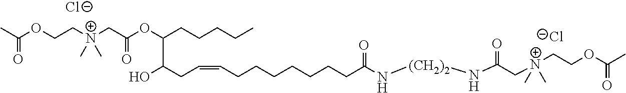 |
| GLH-15 | 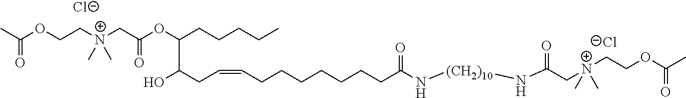 |
| GLH-16 | 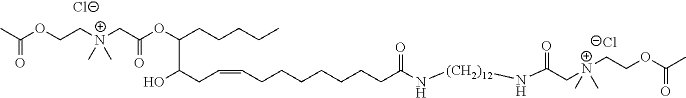 |
| GLH-17 | 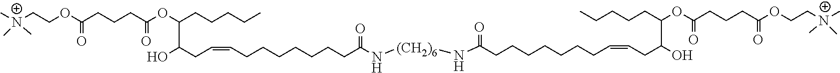 |
| GLH-18 | 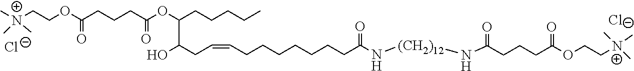 |
| GLH-19 | 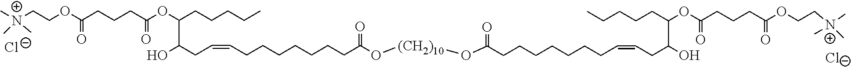 |
| GLH-20 | 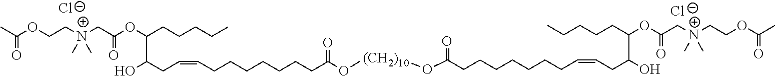 |
| GLH-21 | 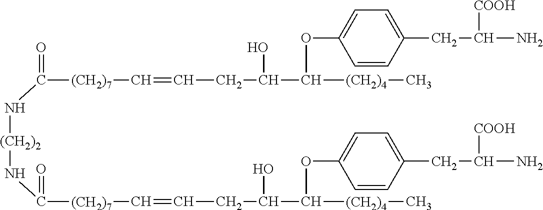 |
| GLH-22 | 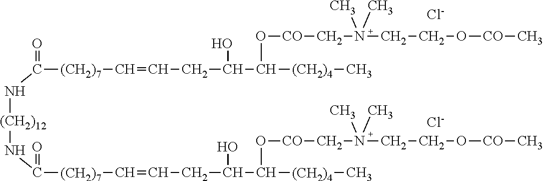 |
| GLH-23 | 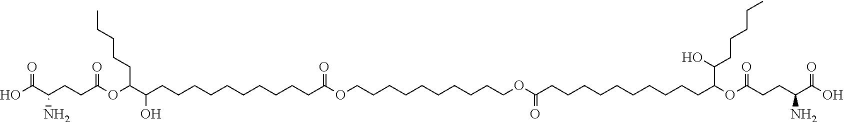 [SMILES: CCCCCC(O)C(CCCCCCCCCCC(=O)OCCCCCCCCCCOC(=O)CCCCCCCCCCC(O)C(CCCCC)OC(=O)CC[C@H](N)C(=O)O)OC(=O)CC[C@H](N)C(=O)O] |
| GLH-24 | 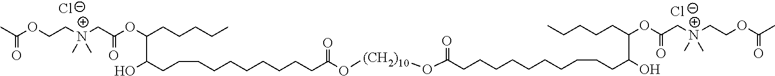 |
| GLH-25 | 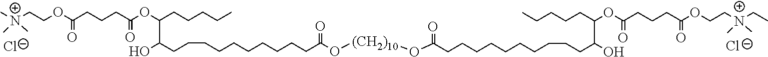 |
| GLH-26 | 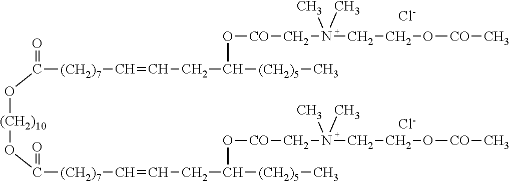 |
| GLH-27 | 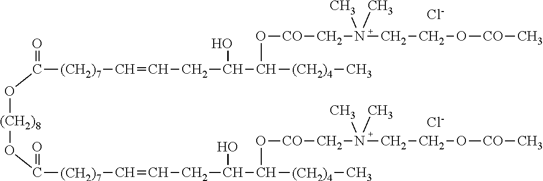 |
| GLH-28 | 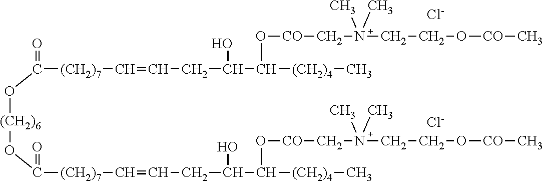 |
| GLH-29 | 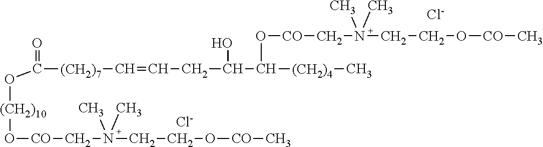 |
| GLH-30 GLH-30 | 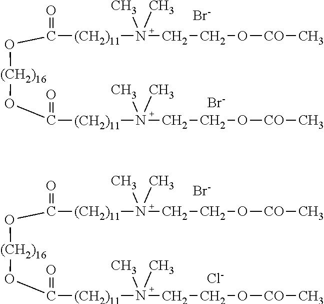 |
| GLH-31 | 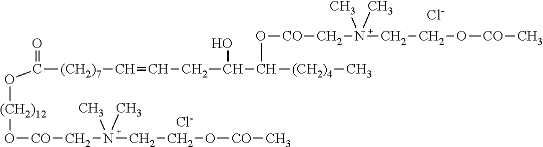 |
| GLH-32 | 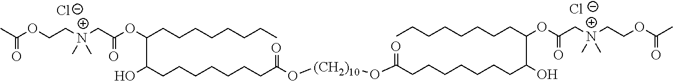 |
| GLH-33 | 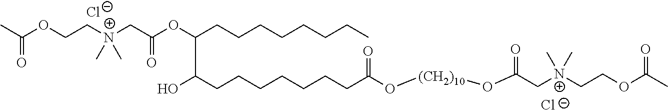 |
| GLH-34 | 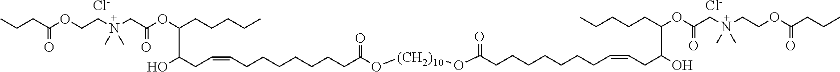 |
| GLH-35 | 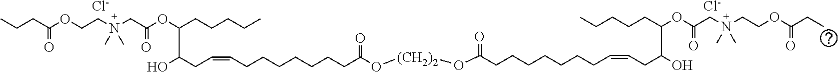 |
| GLH-36 | 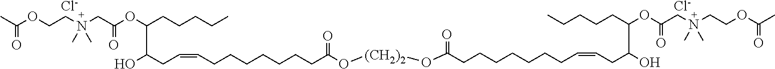 |
| GLH-37 | 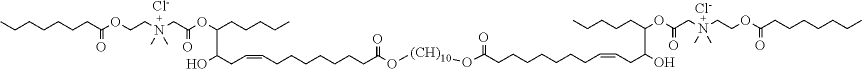 |
| GLH-38 | 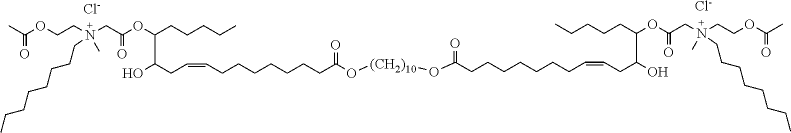 |
| GLH-39^{a} | 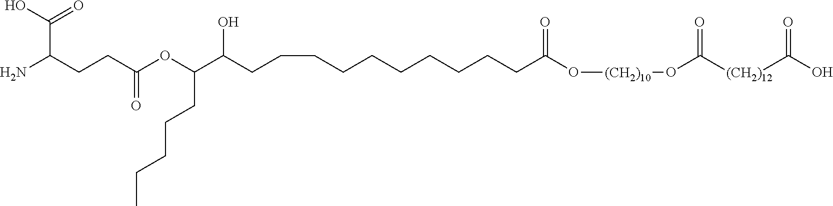 |
| GLH-40 | 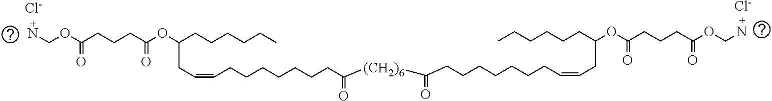 |
| GLH-41 | 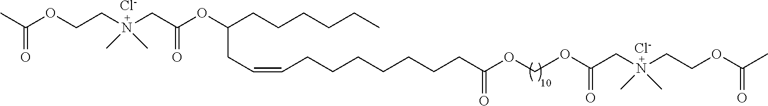 |
| GLH-42^{a} | 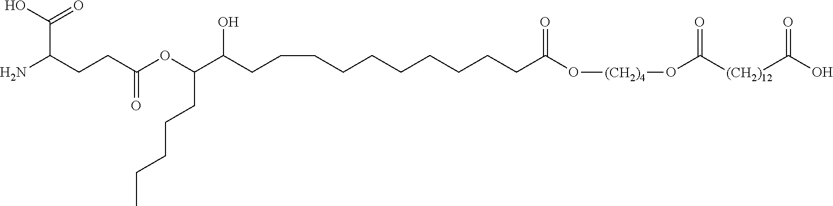 |
| GLH-43^{a} | 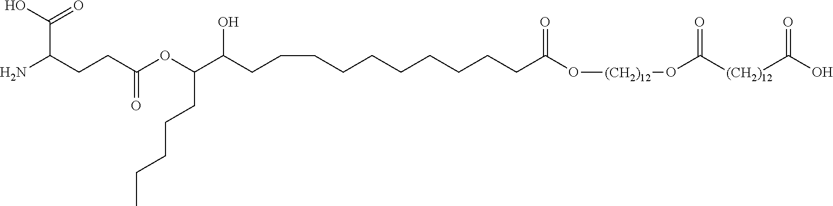 |
| GLH-44 | 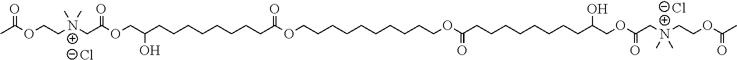 [SMILES: [Cl-] ; CC(=O)OCC[N+](C)(C)CC(=O)OCC(O)CCCCCCCCC(=O)OCCCCCCCCCCOC(=O)CCCCCCCCC(O)COC(=O)C[N+](C)(C)CCOC(C)=O ; [Cl-]] |
| GLH-45 | 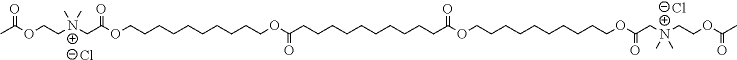 [SMILES: [Cl-] ; CC(=O)OCC[N+](C)(C)CC(=O)OCCCCCCCCCCOC(=O)CCCCCCCCCCC(=O)OCCCCCCCCCCOC(=O)C[N+](C)(C)CCOC(C)=O ; [Cl-]] |
| GLH-46 | 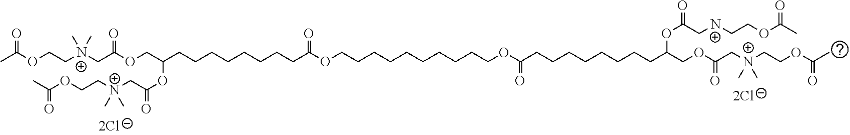 |
| GLH-47 | 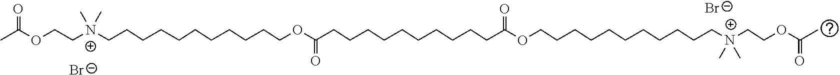 [SMILES: [Br-] ; CC(=O)OCC[N+](C)(C)CCCCCCCCCCCOC(=O)CCCCCCCCCCC(=O)OCCCCCCCCCCC[N+](C)(C)CCOC(C)=O ; [Br-]] |
| GLH-48 | 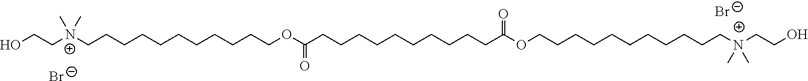 [SMILES: [Br-] ; C[N+](C)(CCO)CCCCCCCCCCCOC(=O)CCCCCCCCCCC(=O)OCCCCCCCCCCC[N+](C)(C)CCO ; [Br-]] |
| GLH-49^{a} | 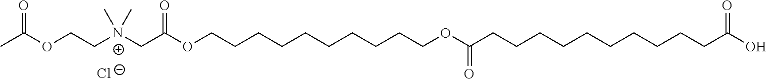 [SMILES: CC(=O)OCC[N+](C)(C)CC(=O)OCCCCCCCCCCOC(=O)CCCCCCCCCCC(=O)O ; [Cl-]] |
| GLH-50^{a} | 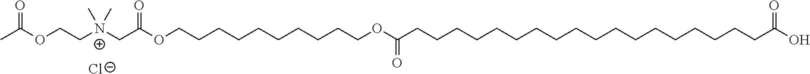 [SMILES: CC(=O)OCC[N+](C)(C)CC(=O)OCCCCCCCCCCOC(=O)CCCCCCCCCCCCCCCCCCC(=O)O ; [Cl-]] |
| GLH-51^{a} | 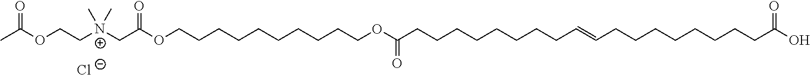 [SMILES: CC(=O)OCC[N+](C)(C)CC(=O)OCCCCCCCCCCOC(=O)CCCCCCCC/C=C/CCCCCCCCC(=O)O ; [Cl-]] |
| GLH-52^{a} | 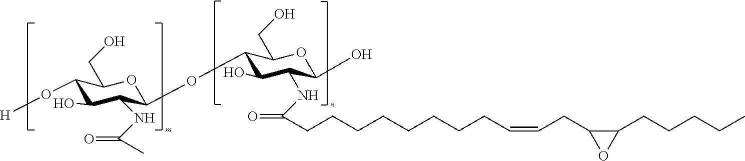 |
| GLH-53^{a} | 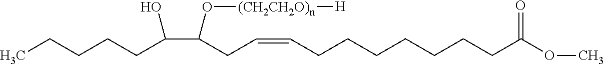 |
| GLH-54^{a} | 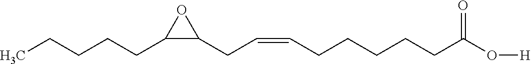 [SMILES: CCCCCC1OC1C/C=C\CCCCCC(=O)O] |
| GLH-55 | 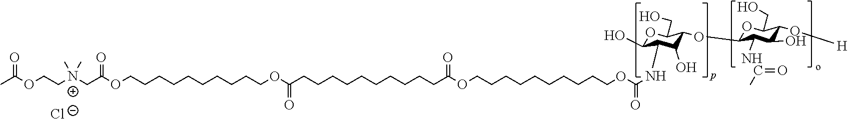 |
| GLH-56 | 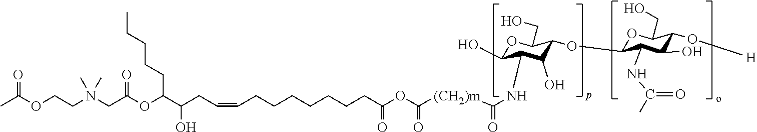 |
| 1 mgGLH- 57 | 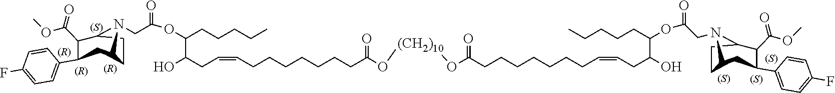 |

^{a} — an intermediate

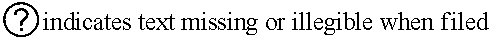

### Example 2

### Formation of Bolaamphiphiles/siRNA Complex

Vesicles are prepared by dissolving 10 mg/ml bolaamphiphile in chloroform together with 2.1 mg/ml cholesteryl hemisuccinate and 1.6 mg/ml cholesterol. The organic solvent is evaporated under nitrogen and then is kept under vacuum overnight. The thin film that was formed is hydrated by RNAs-free water to a concentration of 10 mg/ml of the bolaamphiphile and the suspension which was formed after mixing is sonicated to form 100 nm vesicles at a concentration of 10 mg/ml of bolaamphiphile. These vesicles are used to form the complex with the siRNA duplex as described below.

The siRNA duplex is mixed with pre-prepared vesicles (concentration of the siRNA ranges between 100 nM and 10 μM, and the concentration of the bolaamphiphile ranges between 200 μg/ml and 1 mg/ml). The vesicles that are prepared at a concentration of the bolaamphiphile of 10 mg/ml are diluted before mixing to a concentration that ranges between 200 μg/ml and 1 mg/ml. The mixture is allowed to stand on ice for 30 min-24 hours.

### Example 3

### Transfection of Cell Cultures with Bolaamphiphile/siRNA Complexes

All transfections are performed using bolaamphiphiles of the invention. The concentration of siRNA (200 nM-10 μM) to bolaamphiphiles (200 μg/ml-1 mg/ml) can be 10×-1000×. The transfection is done with either eGFP siRNA that can silence the eGFP gene, which is expressed by the transfected cells. Alternatively, to determine if the siRNA penetrated into the cells, the inventors used siRNA-fluorescent probe conjugate. The fluorescent probe could be FITC or Alexa Flour such as AF-555. Prior to each transfection, the cell media is swapped with growth medium without serum and the prepared siRNA/bolaamphiphile vesicles complexes (as described in Example 2) are diluted to the final concentration of 1×. The cells are incubated for 5-12 hours followed by changing the media to the growth medium.

### Example 4

### Fluorescent Light Microscopy

To assess the silencing efficiency, or the number of the fluorescent cells in the case where non-fluorescent cells were transfected by siRNA-fluorescent probe conjugate, cells are imaged 72 hours after the transfection or 5-24 hours after the transfection, respectively, with a fluorescent microscope.

### Example 5

### Imaging Isolated Organs after Administration of siRNA/Bolaamphiphile Vesicle Complex to Mice

siRNA/bolaamphiphile vesicles complex, prepared by mixing siRNA-AF555 conjugate with bolaamphiphilic vesicles (as described in example 2 above), was injected intravenously to mice via the tail vein. The injected dose ranged between 20 mg/kg to 40 mg/kg of the bolaamphiphile. Mice were sacrificed 30 min and 2 hours after the injection and organs were washed by saline and fluorescence imaging of the isolated organs was performed.

Based on the results, it can be determined that bolaamphiles have the potential to be used as the carriers for siRNA delivery. Bola/siRNA vesicle complexes significantly increase the stability of siRNAs, provided resistence against nucleases activity and provide excellent intracellular uptake followed by a specific gene silencing. Moreover, depending on application, the extent of protection of siRNA can be altered by simply changing the carrier.

### Example 6

### Bolaamphiphiles with a Histidine Head Group

A bolaamphiphile similar to the structure of GLH-19 is formed wherein, instead of the binding of the acetylchloline head groups through the amino group, a histidine is used instead and the histidine is bound through its alpha-amino group. The synthesis of this bolaamphiphilic is carried out in accordance with the methods described previously (Chemistry and Physics of Lipids 2008, 153, 85-97; Journal of Liposome Research 2010, 20, 147-59; WO2002/055011; WO2003/047499; and/or WO2010/128504) and methods known in the art using reagents, starting materials, and purification methods known to those skilled in the art.

More specifically, vesicles are prepared by dissolving 10 mg/ml of the histidine bolaamphiphile in chloroform together with 2.1 mg/ml cholesteryl hemisuccinate and 1.6 mg/ml cholesterol. The organic solvent is evaporated under nitrogen and then is kept under vacuum overnight. The thin film that is formed is hydrated in water at pH 6.8, to a concentration of 10 mg/ml of the bolaamphiphile and the suspension formed after mixing is sonicated to form 100 nm vesicles at a concentration of 10 mg/ml of bolaamphiphile. These vesicles are stable at the concentration formed and upon dilution and show very good shelf like. When placed in pH 4-5 solution they disrupt upon protonation of the Nitrogen of the imidzoale ring, releasing an encapsulated marker such as carboxyfluorescein.

### Example 7

### Formation of Bolaamphiphiles mRNA Complex

Vesicles are prepared by dissolving 10 mg/ml bolaamphiphile (GLH 19 and GLH 20 in a ratio of 2/1) in chloroform together with 2.1 mg/ml cholesteryl hemisuccinate and 1.6 mg/ml cholesterol. The organic solvent is evaporated under nitrogen and then is kept under vacuum overnight. The thin film that is formed is hydrated by a mRNAs-water mixture to a concentration of 10 mg/ml of the bolaamphiphile and the suspension which is formed after mixing is sonicated to form 100 nm vesicles at a concentration of 10 mg/ml of bolaamphiphile. These vesicles are used to form the complex with the mRNA duplex as described below. In this example both 1.8 kB transcript and a 6.2 kB mRNA are successfully encapsulated.

The mRNA duplex is mixed with pre-prepared vesicles (concentration of the mRNA ranges between 100 nM and 10 μM, and the concentration of the bolaamphiphile ranges between 200 μg/ml and 1 mg/ml) with 60 to 90% encapsulation efficiency as a function of the mRNA concentration, the mRNA molecular weight, and the bolaamphiphile concentrations. The vesicles that are prepared at a concentration of the bolaamphiphile of 10 mg/ml are diluted before mixing to a concentration that ranges between 200 μg/ml and 1 mg/ml. The mixture is allowed to stand on ice for 30 min-24 hours.

These vesicles have a 60 to 90% encapsulation of the particular mRNA used, with the veseicles having an average diameter of between 80 to 90 nano-meters (nm) and a cationic surface charge.

### Example 8

### Delivery of mRNA to the Liver with Bolaamphiphile Vesicles

Nano-vesicle complexes prepared in the above examples are used to encapsulate different mRNA for delivery to hepatocyte cells of the liver. As a function of the mRNA encapsulated they are shown to be efficacious in the prevention and treatment of diseases in which the following processes are involved: protein synthesis, protein storage, carbohydrate transformation, synthesis of cholesterol, bile salts, and phospholipids, and detoxification, modification, and excretion of exogenous and endogenous substances.

The same formulations also showed good BBB penetration and uptake into the CNS. Uptake in the CNS and penetration through biological barriers such as the BBB are improved in vesicles formulated with bolaamphiphiles with at least one head group being chitosan such as GLH 55a and are added together with the GLH 19 and GLH 20 in a range of about, but not limited to 1 mg/ml. In each preparation described above, the vesicles are stable and can protect the nucleotides from nucleases. Upon disruption by head group hydrolysis or alteration the nucleotides are released in a fully-active form.

### Example 9

### Delivery of mRNA to the Liver Using Histidine Head Group Containing Vesicles

Bolaamphiphiles having the histine head groups, (Example 6) are used instead of GLH 19 and GLH 20 (as in Exampled 7 and 8) for encapsulation of mRNA. Vsicles of about 100 nm are achieved which show good cell uptake in hepatocte cells and relelase of an active agent within these cells.

### Example 10

### Encapsulation and Delivery of Antisense Oligonucleotides

Vesicle formation and encapsulation are carried out as in Example 8, above, but using antisense oligonucleotides instead of mRNA as the active agent being encapsulated. Vesicle—both with and without chitosan bolaamphilies (e.g., GLH 55)—are prepared and have an average vesicle complexant size of 60 to 110 nm. The vesicles show good transfection into cells and intact penetration across biological barriers such as the BBB and delivery into the brain. The vesicles are stable and can protect the oligonucleotides from nucleases. Upon disruption by head group hydrolysis or alteration, the antisense oligo nucleotides are released in a fully-active form.

### Example 11

### Bolaamphiphile Vesicles Comprising Peptide Head Groups

Vesicle formation and encapsulation are carried out as in Example 8, above, in compositions of GLH 19 and GLH 20 using the chitosan bolaamphiphile GLH 55, and in addition, with a bolaamphiphile having a head group comprising the novel peptide described in Neurobiol Dis. 2005 August; 19(3):407-18 by Liu et al., a peptide having the binding characteristics of tetanus toxin. These vesicle complexes are used to encapsulate GDNF (0.2 mg/10 ml) with an encapsualtion of 90% in vesicles of ˜100 nm diameter. The GDNF remains active when encapsulated and upon release upon vesicle decapsulation after vesicle surface bolaamphiphile head group hydrolysis. When injected into mouse models of amyotrophic lateral sclerosis (ALS) they are shown to have significantly better efficacy as compared to control vesicles without this peptide ligand.

### Example 12

### Bolaamphiphile Vesicles Comprising Tet-1 Peptide Head Groups

Example 11 is repeated except that the boloaamphiphile head group is Tet1, a novel 12 amino acid peptide, that is used for targeting neurotherapeutic proteins instead of the targeting peptide used in Example 11. Equally good efficacy is shown (as compared to vesicles of Example 11 comprising peptide having the binding characteristics of tetanus toxin as head group) which efficiency is significantly better than as seen with vesciles without the peptide ligand.

From the foregoing description, various modifications and changes in the compositions and methods provided herein will occur to those skilled in the art. All such modifications coming within the scope of the appended claims are intended to be included therein.

All publications, including but not limited to patents and patent applications, cited in this specification are herein incorporated by reference as if each individual publication were specifically and individually indicated to be incorporated by reference herein as though fully set forth.

At least some of the chemical names of compounds of the invention as given and set forth in this application, may have been generated on an automated basis by use of a commercially available chemical naming software program, and have not been independently verified. Representative programs performing this function include the Lexichem naming tool sold by Open Eye Software, Inc. and the Autonom Software tool sold by MDL, Inc. In the instance where the indicated chemical name and the depicted structure differ, the depicted structure will control.

Chemical structures shown herein were prepared using ISIS®/DRAW. Any open valency appearing on a carbon, oxygen or nitrogen atom in the structures herein indicates the presence of a hydrogen atom. Where a chiral center exists in a structure but no specific stereochemistry is shown for the chiral center, both enantiomers associated with the chiral structure are encompassed by the structure.

### REFERENCES

1. Afonin, K. A. et al. Self-assembly of functionalized RNA nanoparticles demonstrating potential advancements in automated nanomedicine. Nat Protoc (2011).
2. Davis, M. E. The first targeted delivery of siRNA in humans via a self-assembling, cyclodextrin polymer-based nanoparticle: from concept to clinic. Mol Pharm 6, 659-668 (2009).
3. Davis, M. E. et al. Evidence of RNAi in humans from systemically administered siRNA via targeted nanoparticles. Nature 464, 1067-1070 (2010).
4. Kole, R., Krainer, A. R. & Altman, S. RNA therapeutics: beyond RNA interference and antisense oligonucleotides. Nature reviews 11, 125-140 (2012).
5. Pecot, C. V., Calin, G. A., Coleman, R. L., Lopez-Berestein, G. & Sood, A. K. RNA interference in the clinic: challenges and future directions. Nat Rev Cancer 11, 59-67 (2011).
6. Berkhout, B. & Sanders, R. W. Molecular strategies to design an escape-proof antiviral therapy. Antiviral Res (2011).
7. Grabow, W. W. et al. Self-assembling RNA nanorings based on RNAI/II inverse kissing complexes. Nano Lett 11, 878-887 (2011).
8. Guo, P. The emerging field of RNA nanotechnology. Nat Nanotechnol 5, 833-842 (2010).
9. Shu, Y., Cinier, M., Shu, D. & Guo, P. Assembly of multifunctional phi29 pRNA nanoparticles for specific delivery of siRNA and other therapeutics to targeted cells. Methods 54, 204-214 (2011).
10. Shukla, G. C. et al. A Boost for the Emerging Field of RNA Nanotechnology. ACS Nano 5, 3405-3418 (2011).
11. Hannon, G. J. & Rossi, J. J. Unlocking the potential of the human genome with RNA interference. Nature 431, 371-378 (2004).
12. Fire, A. et al. Potent and specific genetic interference by double-stranded RNA in Caenorhabditis elegans. Nature 391, 806-811 (1998).
13. Elbashir, S. M., Lendeckel, W. & Tuschl, T. RNA interference is mediated by 21- and 22-nucleotide RNAs. Genes Dev 15, 188-200 (2001).
14. Elbashir, S. M., Martinez, J., Patkaniowska, A., Lendeckel, W. & Tuschl, T. Functional anatomy of siRNAs for mediating efficient RNAi in Drosophila melanogaster embryo lysate. Embo J 20, 6877-6888 (2001).
15. Foged, C. siRNA delivery with lipid-based systems: promises and pitfalls. Current topics in medicinal chemistry 12, 97-107 (2012).
16. Vader, P., van der Aa, L. J., Storm, G., Schiffelers, R. M. & Engbersen, J. F. Polymeric carrier systems for siRNA delivery. Current topics in medicinal chemistry 12, 108-119 (2012).
17. Schroeder, A., Levins, C. G., Cortez, C., Langer, R. & Anderson, D. G. Lipid-based nanotherapeutics for siRNA delivery. Journal of internal medicine 267, 9-21 (2010).
18. Grinberg, S., Kipnis, N., Linder, C., Kolot, V. & Heldman, E. Asymmetric bolaamphiphiles from vernonia oil designed for drug delivery. Eur. J. Lipid Sci. Technol. 112, 137-151 (2010).
19. Grinberg, S. et al. Novel cationic amphiphilic derivatives from vemonia oil: synthesis and self-aggregation into bilayer vesicles, nanoparticles, and DNA complexants. Langmuir 21, 7638-7645 (2005).
20. Dakwar, G. R. et al. Delivery of proteins to the brain by bolaamphiphilic nano-sized vesicles. J Control Release 160, 315-321 (2012).
21. Wiesman, Z. et al. Novel cationic vesicle platform derived from vernonia oil for efficient delivery of DNA through plant cuticle membranes. Journal of biotechnology 130, 85-94 (2007).
22. Linder, C., Grinberg, S. & Heldman, E. Nano-sized particles comprising multi-headed amphiphiles for targeted drug delivery. PCT patent application PCT/IL2010/000359, WO/2010/128504, (2010).
23. Popov, M. et al. Site-directed decapsulation of bolaamphiphilic vesicles with enzymatic cleavable surface groups. J Control Release 160, 306-314 (2012).
24. Bindewald, E., Afonin, K., Jaeger, L. & Shapiro, B. A. Multistrand RNA secondary structure prediction and nanostructure design including pseudoknots. ACS Nano 5, 9542-9551 (2011).
25. Kim, D. H. et al. Synthetic dsRNA Dicer substrates enhance RNAi potency and efficacy. Nat Biotechnol 23, 222-226 (2005).
26. Rose, S. D. et al. Functional polarity is introduced by Dicer processing of short substrate RNAs. Nucleic Acids Res 33, 4140-4156 (2005).
27. Afonin, K. A. et al. In vitro assembly of cubic RNA-based scaffolds designed in silico. Nat Nanotechnol 5, 676-682 (2010).

1.-86. (canceled)

87. A pharmaceutical composition or a formulation comprising a bolaamphiphile vesicle complex; wherein the bolaamphiphile vesicle complex comprises one or more bolaamphiphilic compounds and at least one biologically-active compound selected from the group consisting of an mRNA molecule, an antisense oligonucleotide, a natural or synthetic peptide or proteins, and a combination of two or more thereof, and
wherein the bolaamphiphilic compound is a compound according to formula I:
HG²-L¹-HG¹ I
or a pharmaceutically acceptable salt, solvate, hydrate, prodrug, stereoisomer, tautomer, isotopic variant, or N-oxide thereof, or a combination thereof;
wherein:
each HG¹ and HG² is independently a hydrophilic head group; and
L¹ is alkylene, alkenyl, heteroalkylene, or heteroalkenyl linker; unsubstituted or substituted with C₁-C₂₀ alkyl, hydroxyl, or oxo.

88. A pharmaceutical composition of claim 87, wherein
L¹ is heteroalkylene, or heteroalkenyl linker comprising C, N, and O atoms; unsubstituted or substituted with C₁-C₂₀ alkyl, hydroxyl, or oxo.

89. A pharmaceutical composition of claim 87, wherein the bolaamphiphilic compound is a compound according to formula II, III, IV, V, or VI: 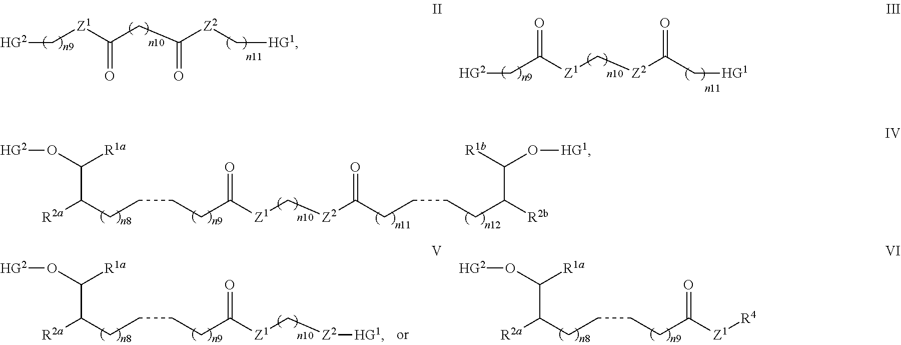
or a pharmaceutically acceptable salt, solvate, hydrate, prodrug, stereoisomer, tautomer, isotopic variant, or N-oxide thereof, or a combination thereof;
wherein:
each HG¹ and HG² is independently a hydrophilic head group;
each Z¹ and Z² is independently —C(R³)₂—, —N(R³)— or —O—;
each R^{1a}, R^{1b}, R³, and R⁴ is independently H or C₁-C₈ alkyl;
each R^{2a} and R^{2b} is independently H, C₁-C₈ alkyl, OH, alkoxy, or O-HG¹ or O-HG²;
each n8, n9, n11, and n12 is independently an integer from 1-20;
n10 is an integer from 2-20; and
each dotted bond is independently a single or a double bond.

90. A pharmaceutical composition of claim 89, wherein the bolaamphiphilic compound is a compound according to formula II, III, IV, V, or VI; and
each R^{1a} and R^{1b} is independently H, Me, Et, n-Pr, i-Pr, n-Bu, i-Bu, sec-Bu, n-pentyl, isopentyl, n-hexyl, n-heptyl, or n-octyl.

91. A pharmaceutical composition of claim 89, wherein the bolaamphiphilic compound is a compound according to formula II, III, IV, V, or VI; and each HG¹ and HG² is independently selected from: 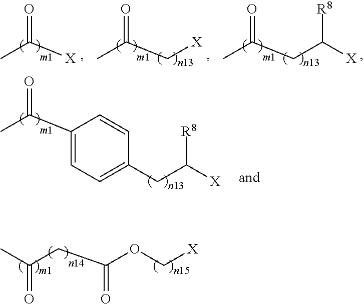
wherein:
X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocycle;
each R^{5c} is independently substituted or unsubstituted C₁-C₂₀ alkyl; each R⁸ is independently H, substituted or unsubstituted C₁-C₂₀ alkyl, alkoxy, or carboxy;
m1 is 0 or 1; and
each n13, n14, and n15 is independently an integer from 1-20.

92. A pharmaceutical composition of claim 89, wherein the bolaamphiphilic compound is a compound according to formula VIIa, VIIb, VIIc, or VIId: 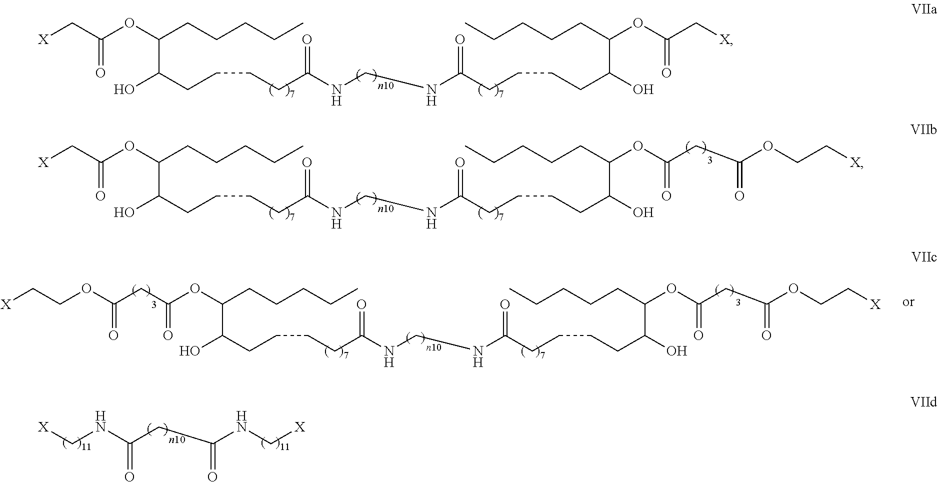
or a pharmaceutically acceptable salt, solvate, hydrate, prodrug, stereoisomer, tautomer, isotopic variant, or N-oxide thereof, or a combination thereof;
wherein:
each X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocycle;
each R^{5c} is independently substituted or unsubstituted C₁-C₂₀ alkyl;
n10 is an integer from 2-20; and
each dotted bond is independently a single or a double bond.

93. A pharmaceutical composition of claim 87, wherein the bolaamphiphilic compound is a compound according to formula VIIIa, VIIIb, VIIIc, or VIIId: 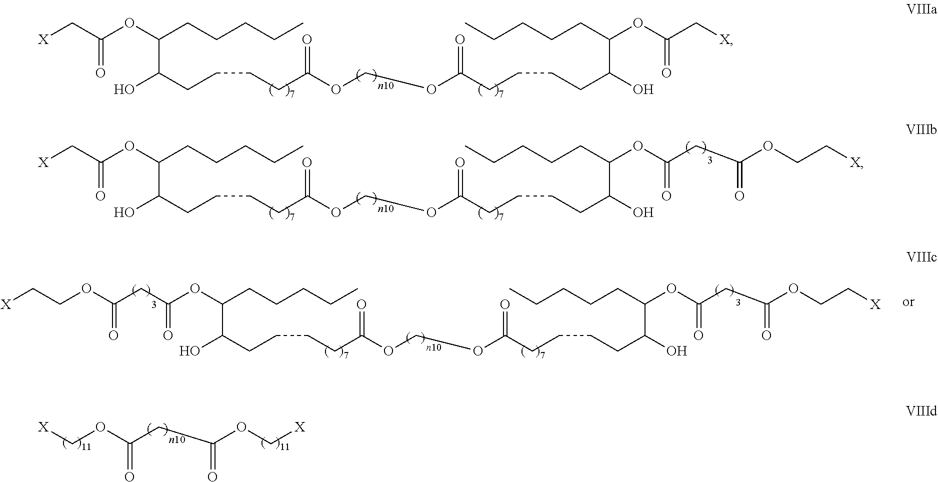
or a pharmaceutically acceptable salt, solvate, hydrate, prodrug, stereoisomer, tautomer, isotopic variant, or N-oxide thereof, or a combination thereof;
wherein:
each X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocycle;
each R^{5c} is independently substituted or unsubstituted C₁-C₂₀ alkyl;
n10 is an integer from 2-20; and
each dotted bond is independently a single or a double bond.

94. A pharmaceutical composition of claim 87, wherein the bolaamphiphilic compound is a compound according to formula IXa, IXb, or IXc: 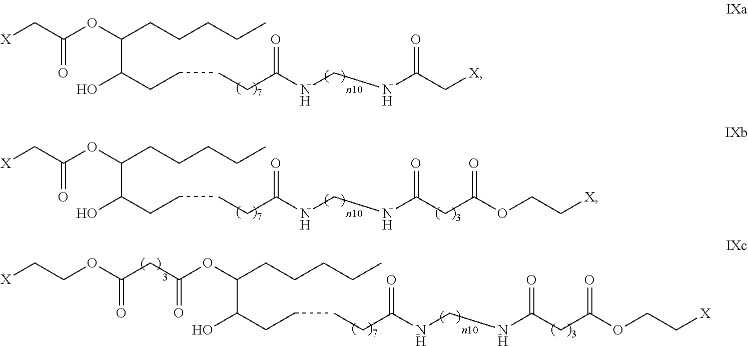
or a pharmaceutically acceptable salt, solvate, hydrate, prodrug, stereoisomer, tautomer, isotopic variant, or N-oxide thereof, or a combination thereof;
wherein:
each X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocycle;
each R^{5c} is independently substituted or unsubstituted C₁-C₂₀ alkyl;
n10 is an integer from 2-20; and
each dotted bond is independently a single or a double bond.

95. A pharmaceutical composition of claim 87, wherein the bolaamphiphilic compound is a compound according to formula Xa, Xb, or Xc: 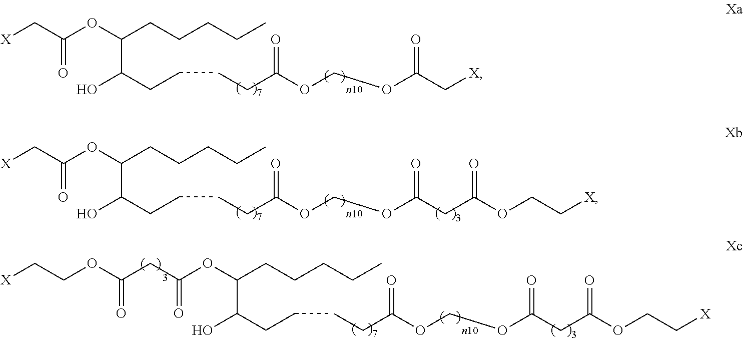
or a pharmaceutically acceptable salt, solvate, hydrate, prodrug, stereoisomer, tautomer, isotopic variant, or N-oxide thereof, or a combination thereof;
wherein:
each X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocycle;
each R^{5c} is independently substituted or unsubstituted C₁-C₂₀ alkyl;
n10 is an integer from 2-20; and
each dotted bond is independently a single or a double bond.

96. A pharmaceutical composition of claim 91, wherein each R^{5a}, R^{5b}, and R^{5c} is independently substituted or unsubstituted C₁-C₂₀ alkyl.

97. A pharmaceutical composition of according to claim 91, wherein X is a chitosanyl group.

98. A pharmaceutical composition of claim 87, wherein the bolaamphiphilic compound is any one of the bolaampiphilic compounds listed in Table 1.

99. A pharmaceutical composition of claim 97, wherein the pharmaceutical composition comprises a pharmaceutically acceptable carrier.

100. A pharmaceutical composition of claim 99, wherein the carrier is a parenteral carrier.

101. A pharmaceutical formulation of claim 87 comprising one or more bolaamphiphilic compounds according to formula I-Xc.

102. A method of delivering at least one biologically-active compound selected from the group consisting of an mRNA molecule, an antisense oligonucleotide, a natural or synthetic peptide or proteins, and a combination of two or more thereof, into a non-human animal cell or a human cell comprising the step of administering to the animal or human a pharmaceutical composition comprising of claim 87.

103. A method of claim 102, wherein the cell is a brain cell, liver cell, gall bladder cell, a lung cell, a cell of a lymph node, a CD4+ lymphocyte, a cell of the mononuclear phagocyte system, a monocyte, macrophage, a resident brain microglial cell, or a dendritic cell.

104. A nano-particle comprising one or more bolaamphiphilic compounds of claim 87 and a biologically-active compound selected from the group consisting of an mRNA molecule, an antisense oligonucleotide, a peptide targeting ligand, and a combination of two or more thereof.

105. A nano-particle of claim 104, wherein the bolaamphiphilic compounds and at least one biologically-active compound selected from the group consisting of an mRNA molecule, and an antisense oligonucleotide are encapsulated within the nano-particle.

106. A nano-sized particle of claim 105 comprising mRNA and a pharmaceutically acceptable carrier.

107.-109. (canceled)

110. A pharmaceutical composition of claim 87, wherein the nano-vesicles comprises a surface decorated with peptides having the binding characteristics of tetanus toxin.

111. A pharmaceutical composition of claim 87, wherein the biologically-active compound is an enkephalin, insulin, insulin analogs, oxytocin, calcitonin, tyrotropin releasing hormone, follicle stimulating hormone, luteinizing hormone, vasopressin, vasopressin analog, catalase, interleukin-II, interferon, colony stimulating factor, tumor necrosis factor (TNF), melanocyte-stimulating hormone, superoxide dismutase, glial cell derived neurotrophic factor (GDNF), a Gly-Leu-Phe (GLF) family member, an RNA duplex, an RNA-DNA duplex, DNA plasmid, an antiviral agent, an antibacterial agent, an antineoplastic agent, a chemotherapy agent, and a topoisomerase inhibitor.

112. A pharmaceutical composition of claim 87, wherein at least one linkage of the mRNA and antisense oligonucleotide is a stable non-phosphodiester linkage.

113. A pharmaceutical composition of claim 112, wherein the stable non-phosphodiester linkage is a phosphorothioate, phosphorodithioate, phosphoroselenate, methylphosphonate, or O-alkyl phosphotriester linkage.

114. A pharmaceutical composition of claim 111, wherein the biologically-active compound is selected from the group consisting of adriamycin, angiostatin, azathioprine, bleomycin, busulfane, camptothecin, carboplatin, carmustine, chlorambucile, chlormethamine, chloroquinoxaline sulfonamide, cisplatin, cyclophosphamide, cycloplatam, cytarabine, dacarbazine, dactinomycin, daunorubicin, didox, doxorubicin, endostatin, enloplatin, estramustine, etoposide, extramustinephosphat, flucytosine, fluorodeoxyuridine, fluorouracil, gallium nitrate, hydroxyurea, idoxuridine, leuprolide, lobaplatin, lomustine, mannomustine, mechlorethamine, mechlorethaminoxide, melphalan, mercaptopurine, methotrexate, mithramycin, mitobronitole, mitomycin, mycophenolic acid, nocodazole, oncostatin, oxaliplatin, paclitaxel, pentamustine, platinum-triamine complex, plicamycin, prednisolone, prednisone, procarbazine, protein kinase C inhibitors, puromycine, Semustine, signal transduction inhibitors, spiroplatin, streptozotocine, stromelysin inhibitors, taxol, tegafur, telomerase inhibitors, teniposide, thalidomide, thiamiprine, thioguanine, thiotepa, tiamiprine, tretamine, triaziquone, trifosfamide, tyrosine kinase inhibitors, uramustine, vidarabine, vinblastine, vinca alcaloids, vincristine, vindesine, vorozole, zeniplatin, zeniplatin, zinostatin, irinotecan, topotecan, and combinations of two or more thereof.

115. A pharmaceutical formulation of claim 87, wherein at least one bolaamphiphile comprises a head group selected from the group consisting of choline, thiocholine, O-alkyl choline, N-alkyl choline, and a choline ester derivatives thereof, glutamic acid, aspartic acid, lysine, cysteine, tyrosine, tryptophan, phenylalanine, levodopa (3,4-dihydroxy-phenylalanine), p-aminophenylalanine, a peptidase substrate, enkephalin, N-acetyl-ala-ala, a peptide comprising a domain recognized by beta and gamma secretases, a peptide comprising a domain recognized by stromelysins, a saccharide, glucose, mannose, ascorbic acid, nicotine, cytosine, lobeline, polyethylene glycol, a cannabinoid, and folic acid.

116. A pharmaceutical formulation of claim 87, wherein at least one bolaamphiphile comprises a histidine head group.

117. A pharmaceutical formulation of claim 87, wherein at least one bolaamphiphile comprises a head group comprising a Tet 1 peptide.

118. A pharmaceutical formulation of claim 87, wherein the bolaamphiphile complexes further comprise at least one additive selected from the group consisting of cholesterol, a neutral, cationic or anionic cholesterol derivative, cholesterol hemisuccinate, cholesterol oleyl ether, a single headed amphiphile with one, two or multiple aliphatic chains, phospholipids, a zwitterionic, acidic, or cationic lipid, phosphatidylcholine, phosphatidylethanol amine, sphingomyelin, a phosphatidylglycerol, a phosphatidylserine, a phosphatidylinositol, a phosphatidic acid, diacyl trimethylammonium propane, diacyl dimethylammonium propane, and stearylamine, a cationic amphiphile, spermine cholesterol carbamate, and chitosan.
